(19) United States
(12) Patent Application Publication
(10) Pub. No.: US 2026/0101858 A1
(43) Pub. Date: Apr. 16, 2026

(54) PLANT TISSUE CULTURE DEVICES AND METHODS

(71) Applicant: Rebel Cultures, LLC, West Olive, MI (US)

(72) Inventors: Della Fetzer, West Olive, MI (US); Rachel Carrie VonDestinon, Spring Lake, MI (US)

(21) Appl. No.: 19/420,713

(22) Filed: Dec. 15, 2025

Related U.S. Application Data
(63) Continuation of application No. PCT/US24/37605, filed on Jul. 11, 2024.
(60) Provisional application No. 63/526,286, filed on Jul. 12, 2023.

(51) Int. Cl. A01H 4/00

(52) U.S. Cl. CPC A01H 4/001

(57) ABSTRACT

In some embodiments, the techniques described herein relate to systems and methods for plantlet propagation. A plant tissue culture apparatus can include: a replication capsule that can contain a plurality of plantlets; a light source to illuminate the plurality of plantlets; a first gas tank coupled to the replication capsule containing a first gas; a first liquid tank coupled to the replication capsule contain a first liquid mixture; a nanobubble generator positioned between the first liquid tank and the liquid inlet; and a second gas tank containing a second gas, where the second gas tank is coupled to the nanobubble generator, and the nanobubble generator is configured to generate bubbles including the second gas in the first liquid mixture. The system can also include a processor configured to control valves and a light source of the system based on predetermined parameters or in response to information from a sensor.

### CROSS-REFERENCE TO RELATED APPLICATIONS

This application is a continuation of International Patent Application Ser. No. PCT/US24/37605, filed Jul. 11, 2024 and published as WO 2025/015166, which claims the priority benefit of U.S. Provisional Patent Application Ser. No. 63/526,286, filed Jul. 12, 2023, the contents of which are herein incorporated by reference in their entirety.

### INCORPORATION BY REFERENCE

All publications and patent applications mentioned in this specification are herein incorporated by reference in their entirety, as if each individual publication or patent application was specifically and individually indicated to be incorporated by reference in its entirety.

### TECHNICAL FIELD

This disclosure relates generally to the field of biotechnology, and more specifically to the fields of horticultural and agricultural propagation. Described herein are plant tissue culture devices and methods.

### BACKGROUND

Plant tissue culture techniques are widely used in agriculture and horticulture to rapidly produce vigorous and mature genetic clones from a desired explant which are free of pathogens due to the aseptic environment in which they are developed and propagated. Conventional plant tissue culture plantlets are developed within sterilized containers containing a specialized semi-solid gel or liquid nutrient media and housed on shelves under lighting within a laboratory. Propagation is achieved when sterile surgical tools are used to strategically divide plantlets using aseptic procedures, most often within a laminar flow hood. Propagated plantlets are transferred to a new sterile vessel with nutrient media and returned to the lighted shelving area where development of new plantlets or root tissue is stimulated by the nutrient media over a period of several weeks. Subsequently, such a propagation cycle can be repeated, or the plantlet can be transferred to a greenhouse for further development.

### SUMMARY

In some embodiments, the techniques described herein relate to a plant tissue culture apparatus including: a replication capsule including a capsule body, liquid inlets, gas inlets, a gas outlet, and a liquid outlet, wherein an internal volume defined by the capsule body is configured to contain a plurality of plantlets; a light source configured to illuminate the plurality of plantlets in the replication capsule; a first gas tank coupled to the gas inlet and configured to contain a first gas; a first liquid tank coupled to the liquid inlet, the first liquid tank configured to contain a first liquid mixture; a nanobubble generator positioned between the first liquid tank and the liquid inlet; a second gas tank configured to contain a second gas, wherein the second gas tank is coupled to the nanobubble generator, wherein the bubble generator is configured to generate bubbles including the second gas in the first liquid mixture; a liquid valve between the first liquid tank and the liquid inlet; a first gas valve between the first gas tank and the gas inlet; a second gas valve between the second gas tank and the nanobubble generator; and a processor coupled to the liquid valve, the first gas valve, the second gas valve, and the light source, wherein the processor is configured to control the liquid valve, the first gas valve, the second gas valve, and the light source based on predetermined parameters or in response to information from a sensor.

In some embodiments, the techniques described herein relate to a method of propagation of a plantlet including: introducing a first gas from a first gas tank to a gas inlet of a replication capsule using a first gas valve, the replication capsule including a plurality of plantlets; generating bubbles of a second gas in a first liquid mixture using a bubble generator and a second gas tank coupled to the bubble generator using a second gas valve; introducing the first liquid mixture from the bubble generator to a liquid inlet of a replication capsule using a first liquid valve; after a first period of time, draining the first liquid mixture from the replication capsule using a second liquid valve coupled to a liquid outlet of the replication capsule; illuminating the plurality of plantlets with a light source; and controlling the first liquid valve, the first gas valve, the second gas valve, and the light source using a processor coupled to the first liquid valve, the first gas valve, the second gas valve, and the light source, based on predetermined parameters or in response to information from a sensor.

### BRIEF DESCRIPTION OF THE DRAWINGS

The foregoing is a summary, and thus, necessarily limited in detail. The above-mentioned aspects, as well as other aspects, features, and advantages of the present technology are described below in connection with various embodiments, with reference made to the accompanying drawings.

FIG. 1 is a flow chart of a prior art method for culturing plants in vitro.

FIG. 2 is a high-level flow chart of a method for culturing plants in vitro using the devices and methods described herein.

FIG. 3 shows any embodiment of a system for regulating a plant tissue culture system.

FIG. 4 shows a perspective cross-sectional view of an embodiment of a plant tissue culture system.

FIG. 5 shows an embodiment of a modular growth tray design of the plant tissue culture system.

FIG. 6 shows a block diagram of an example system for plant propagation, in accordance with some embodiments.

FIG. 7 shows a schematic example of a system for plant propagation, in accordance with some embodiments.

FIG. 8 shows a schematic example of a system for plant propagation, in accordance with some embodiments.

FIG. 9 shows a schematic example of a system for plant propagation, in accordance with some embodiments.

FIG. 10 shows an example of a replication capsule for plant propagation, in accordance with some embodiments.

FIG. 11 shows a schematic example of a system for plant propagation, including a replication capsule, a liquid tank, and two gas tanks, in accordance with some embodiments.

FIG. 12 shows a schematic example of a system for plant propagation, including multiple replication capsules coupled to one liquid tank, and two gas tanks, in accordance with some embodiments.

FIG. 13 shows a schematic example of a system for plant propagation, including a replication capsule where fresh liquids are introduced from multiple liquid tanks and then collected without being recirculated, in accordance with some embodiments.

FIG. 14 shows a schematic example of a system for plant propagation, including multiple replication capsules, multiple liquid tanks, and two gas tanks, in accordance with some embodiments.

FIG. 15 shows a schematic example of a system for plant propagation from a top view, including multiple replication capsules, and multiple light sources, in accordance with some embodiments.

FIGS. 16A and 16B show an example of a decision tree with feedback loops using the devices, systems, and methods described herein and plantlet photography, in accordance with some embodiments The illustrated embodiments are merely examples and are not intended to limit the disclosure. The schematics are drawn to illustrate features and concepts and are not necessarily drawn to scale.

### DETAILED DESCRIPTION

The foregoing is a summary, and thus, necessarily limited in detail. The above-mentioned aspects, as well as other aspects, features, and advantages of the present technology will now be described in connection with various embodiments. The inclusion of the following embodiments is not intended to limit the disclosure to these embodiments, but rather to enable any person skilled in the art to make and use the contemplated invention(s). Other embodiments may be utilized, and modifications may be made without departing from the spirit or scope of the subject matter presented herein. Aspects of the disclosure, as described and illustrated herein, can be arranged, combined, modified, and designed in a variety of different formulations, all of which are explicitly contemplated and form part of this disclosure.

Many plants for sustaining human life and preserving ecosystems, such as fruit and nut-bearing trees and plants, threatened trees, native plant species, and commercially important ornamental plants, cannot be effectively propagated from seeds due to breeding or seed shortages. Because of these shortcomings, these plants may be propagated by alternative means. Vegetative propagation via nodal or branch cuttings, basal divisions, and bare root segments can be an effective means of propagation for some plant species but uses significant labor and greenhouse infrastructure or field acreage. Further, these propagation methods occur in high-risk environments for disease vectors such as insects, nematodes, and pathogenic microbes and therefore frequently result in plants afflicted by fungal, viral, and intracellular bacterial infections which can transmit to nearby crops, causing widespread agricultural and ecological loss.

An alternative vegetative propagation method, which circumvents these disease vectors, is propagation via plant tissue culture, or micropropagation. Conventional plant tissue culture utilizes laboratory infrastructure, specialized equipment, and highly technical labor to vegetatively propagate young plants in aseptic conditions with sterilized growing containers. This conventional method is an effective propagation method for thousands of plant species which cannot be produced from seed and is valued in horticulture and agriculture for its ability to produce disease-free young plants within less space and within a shorter timeframe than other methods of vegetative propagation. However, the laboratory infrastructure, specialized equipment, and highly technical labor used to maintain an aseptic propagation environment for plant tissue culture are cost-prohibitive for most agricultural, horticultural, and forestry nurseries. Traditional costs associated with laboratory infrastructure, specialized equipment, and highly technical labor are necessitated by the prevention and elimination of bacterial and fungal contaminants. These high costs force nurseries to choose a propagation method with a higher rate of disease transmission or select an external production source which often results in shipping of young plant starts long distances across the country or across the world.

Annually, agricultural producers and governments pay hundreds of billions of dollars to combat the estimated global damages of invasive pests and pathogens. The practice of outsourcing plant propagation increases global exposure to agricultural, environmental, and economic damages. The devices and methods described herein provide a technical solution to the above technical problems by providing a cost-effective method which enables the local propagation of plant tissue culture without laboratory infrastructure, specialized equipment, and highly technical labor. Further, the devices and methods described herein increase plant vitality and phenotypic expression which are known to be positively influenced by the beneficial microbiota found in a plant's natural growing habitat. The inclusion of living microbiota is not possible in laboratory-based plant tissue culture due to the use of aseptic procedures and sterilization of the plant growth medium

The devices, systems, and methods described herein solve the above technical problems with technical solutions. For example, the systems described herein are capable of enabling asexual reproduction of plants using vegetative propagation of young plants in disease-free, non-sterile conditions with beneficial microbiota, and without laboratory infrastructure, specialized equipment, and highly technical labor is used.

A problem with conventional systems is that they require developing plantlets and a propagation container to remain aseptic to avoid uninhibited proliferation of detrimental microbiota. The removal of beneficial microbiota from the plantlet and propagation container can damage the natural defenses of the plant, potentially slowing in vitro growth and proliferation and resulting in a weaker plant ex vitro. In contrast, the devices, systems, and methods described herein use propagation capsules which support and deploy beneficial microbiota throughout the different stages of the in vitro propagation process and can continuously cleanse and balance the microbial landscape within the system.

Conventional systems may use specialized equipment including laminar flow hoods and autoclaves and building infrastructure to prepare sterile culture media and containers and maintain aseptic plantlets in the propagation container. Further, significant technical know-how is required to properly execute the preparation and maintenance of sterile culture media and aseptic plantlets in traditional systems. The requirement of these specialized systems and knowledge prevents current technology from being reasonably deployed by typical agricultural producers who would benefit from rapid production of young, vigorous plants which do not require seed for propagation. To solve the problems of operational complexity and inaccessibility of traditional systems, the devices, systems, and methods described herein can be installed or implemented on any property with water and electricity hookups and can be operated by an unskilled worker (e.g., by simply connecting premixed liquid and gas tanks and/or cartridges). This improves outcomes by subtracting the need for complex ancillary knowledge, equipment, and infrastructure.

Furthermore, in conventional systems, the propagation container represents a static and minimally modifiable internal environment. In contrast, the devices, systems, and methods described herein are designed for automated experimentation with under-studied plant health factors. For example, the devices, systems, and methods described herein can monitor and/or control plant health factors such as temperature, humidity, vapor pressure deficit, light frequency, intensity, and photoperiod, microbial activity, dissolved O₂ and CO₂ concentration, air exchange rate, and modulation of a plurality of gasses, beneficial microbes, hormones, basal salts, and other nutritional biostimulants. Comprehensive modulation of these factors is becoming increasingly necessary for food system and environmental stability amid changing climatic conditions. The devices, systems, and methods described herein are designed to incorporate the modulation and testing of effectiveness of each of these factors in the propagation of robust tissue culture plantlets.

Another drawback of conventional tissue culture is that propagation protocols often require years of trial and error to manually develop because protocol development is not mechanized and automated. This is a problem because high-impact research and commercial outcomes in plant tissue culture are constrained by manual experimental design, which can be biased. In contrast, the devices, systems, and methods described herein are designed to rapidly test a multitude of protocols in parallel by modifying environmental, nutritional, and microbial inputs in parallel replication capsules to progress plantlets toward defined plant health and propagation targets. For example, in vitro plantlet photography and monitoring of waste products exiting the replication capsules can be used to power real-time feedback loops.

Conventional liquid culture systems also typically have a plant culture media that is continuously recirculated without re-sterilization between recirculation cycles. This causes the proliferation of detrimental microbiota including pathogenic fungi, bacteria, and accumulation of biofilm-forming microbes which can be inadvertently introduced into the culture. Once detrimental microbiota is discovered within the propagation containers of traditional systems, the plantlets have already become infected and must be discarded. wherein contrast the devices, systems, and methods described herein include liquid inputs that flow through the system in a single pass before draining or are sanitized before being recirculated.

Another problem with many conventional liquid culture systems, such as those using temporary immersion, is the lack of agitation or rotation of plantlets within the growing container. This is a problem because developing plantlets receive an uneven distribution of light, gas exposure, and nutrients. The technical solution to this problem is a system which incorporates simple mechanical agitation mechanisms, for example, a bubble generator to introduce bubbles into the liquid culture solutions provided to the replication capsule.

Disclosed herein are devices, systems, and methods of plant production using technology which preserves the benefits of conventional plant tissue culture while significantly decreasing costs due to laboratory infrastructure, overhead, and labor as well as decreasing ecological risks of conventional practices while improving the speed and reach of positive research and commercial outcomes in plant propagation. Further, the methods described herein may allow for greater control of the young plant environment, resulting in a higher quality product with reduced crop loss.

The devices, systems, and methods for plant propagation described herein include one or more replication capsules. One or more plantlets can be contained within, or are positionable within, an internal volume of each of the replication capsules, and one or more illumination sources (e.g., including LEDs, one or more incandescent lights, and/or one or more fluorescent lights) is positioned to illuminate the plantlets within the replication capsule. Each replication capsule can include a capsule body containing the internal volume, a liquid inlet, a gas inlet, a gas outlet, and a liquid outlet. In some cases, the liquid inlet and gas inlet can be contained in a cap of the replication capsule. One or more liquid sources can be coupled to the liquid inlet, and one or more gas sources can be coupled to the gas inlet.

The devices, systems, and methods for plant propagation described herein can include automation, where a processor (e.g., processor 33, 35, 37) controls components of the system based on predefined parameters and/or using feedback from one or more sensors. One or more processors (e.g., processor 33, 35, 37) can be used to control the nutrients and/or beneficial microbiota delivered to the plantlets and cleansing agents used to clean the plantlets and replication capsule. Further, one or more processors (e.g., processor 33, 35, 37) can be used to alter a generated bubble gas composition, and/or environmental conditions such as temperature, humidity, and lighting. These parameters can be controlled according to algorithms, set points, acceptable ranges, and programs that can include changing one or more parameters over time. In some cases, the processor (e.g., processor 33, 35, 37) can also integrate feedback from sensors to control the components of the system.

In general, plantlets can experience four stages of development during propagation, including establishment, replication, rooting, and acclimatization. Establishment (or the induction stage) is the adaptation of plant tissue harvested from a selected explant to the in vitro environment. Replication (or the multiplication stage) is the proliferation of or an increase in number of plantlets from the established explant. Rooting (or the root development stage) is the development of root tissue at the base of the plantlets. Acclimatization (or the hardening stage) is a preparation of the plantlets for survival and growth during ex vitro growing by gradually shifting the growing environment. Each of these stages can benefit from different parameters, such as different nutrients delivered to the plantlets, different protocols of beneficial microbiota, different protocols of cleansing agents used to clean the plantlets and/or replication capsule, different generated bubble gas compositions, and/or different environmental conditions such as temperature, humidity, and lighting. The parameters can also be different (and optimized) for different species of plantlet. For example, beneficial microbiota can include individual or naturally occurring consortia of mycorrhiza or dark septate endophytes. For example, cleansing agents can include polyoxyethylene sorbitan (e.g., Tween® 20) or hypochlorous acid.

In some cases, the devices, systems, and methods described herein also include generating bubbles in a liquid using a bubble generator. In some cases, the bubble generator can be a nanobubble generator that generates small bubbles, such as less than a micron in diameter, on average. The liquid containing the bubbles may be introduced into the replication capsule through the liquid inlet. Generating bubbles in the liquid introduced into the replication capsule can be beneficial for the development of the plantlets and for sanitizing and/or decontaminating the replication capsule and exposed surfaces of the plantlets. Bubbles can be used to move plantlets in the replication capsule, which can be beneficial, for example, to expose different parts of the plantlets to light, gas, and nutrients. For example, bubbles in a liquid containing a cleansing agent can facilitate the dispersal of biofilms by providing a physical agitation force on the biofilms. In some cases, the small bubbles are used to provide small scale mechanical agitation, for example to scrub away biofilms, while the larger bubbles are used to provide larger scale mechanical agitation to move plantlets around in the replication capsule. In some cases, the bubbles can contain a gas (e.g., air, carbon dioxide, oxygen, and/or ethylene) that is beneficial to a growing plantlet. In some cases, the bubble generator can enable a higher concentration of the gas in the liquid than would be possible without using the bubble generator. For example, bubbles containing air with excess oxygen, that is air with concentrations of oxygen above that of air (i.e., higher than about 21%, or from about 21% to about 30%, or higher than 30%) can be generated in the liquids delivered to the replication capsules described herein. Oxygen in the bubbles can beneficially cause detrimental microbes to oxidate, and small bubbles (e.g., nanobubbles, which are bubbles with diameters less than about 100 nm) can also physically scrub the plantlets, which can help remove detrimental microbes.

In some cases, the devices, systems, and methods for plant propagation described herein include a non-sterile environment including beneficial or symbiotic microbiota. In contrast to conventional techniques that use sterilized, autoclaved, and/or aseptic enclosures, the devices, systems, and methods for plant propagation described herein do not require sterilization because the symbiotic bacteria help prevent detrimental microbiota from significantly colonizing the replication capsule. The beneficial microorganisms can be symbiotically balanced with the plantlet environment and can discourage or knockdown growth of undesired organisms. The replication capsule can be inoculated with beneficial microbiota, and the environment (e.g., relative humidity, temperature, etc.) can be controlled to be compatible with the beneficial microbiota, such that outside organisms that are harmful to the plantlets can be outcompeted by the cultivated beneficial microbiota. In some cases, a rotation of different microbiota and cleansing agents can be used to prevent detrimental microbes from proliferating in the replication capsule. A dynamic environment with air flowing through the replication capsule can also be used, and the gas in the environment can be controlled using gas tanks coupled to valves controlled by a processor (e.g., processor 33, 35, 37), as described herein.

In some embodiments, the devices, systems, and methods for plant propagation described herein can include self-cleaning systems, such as those including beneficial microbiota and cleansing agents. For example, a cleansing agent can be used between growth cycles when no plantlets are present in the replication capsule. Cleansing agents can include polyoxyethylene sorbitan (e.g., Tween® 20), hypochlorous acid, colloidal silver, or other cleaning agents.

In some embodiments, the devices, systems, and methods for plant propagation described herein include a fan or air compressor coupled to a gas mixer, and one or more gas tanks also coupled to the gas mixer. The gas mixer is coupled to the gas inlet of the replication capsule to provide a gas mixture containing air (provided by the fan or air compressor) and gases from the one or more gas tanks. A series of valves can be used to provide controlled mixtures of gases to the mixer and to the replication capsule. In some cases, the fan or air compressor, and the series of valves can be coupled to a processor (e.g., processor 33, 35, 37), and the processor (e.g., processor 33, 35, 37) can control the valve settings to control the gases provided to the mixer and to the replication capsule.

In some embodiments, the devices, systems, and methods for plant propagation described herein include one or more pressurized gas tanks coupled to a gas mixer, and the gas mixer is coupled to the gas inlet of the replication capsule. In such cases, a gas mixture containing gases from the one or more gas tanks can be provided to the gas inlet of the replication capsule using a series of valves to provide controlled mixtures of gases from the pressurized tanks to the mixer and to the replication capsule. In some cases, the series of valves can be coupled to a processor (e.g., processor 33, 35, 37), and the processor (e.g., processor 33, 35, 37) can control the valve settings to control the gases provided to the mixer and to the replication capsule. Additionally, pressurized tanks can optionally be used in combination with a fan or air compressor coupled to the gas mixer. In such cases, the processor (e.g., processor 33, 35, 37) can control the fan or air compressor and the valve settings to control the gases provided to the mixer and to the replication capsule

In some cases, the devices, systems, and methods for plant propagation described herein include more than one gas inlet. For example, a first gas inlet can be used to provide gases from gas tanks as described above, and a second air inlet can be used to provide ventilation using an air circulation system containing one or more fans or air compressors.

In some cases, the devices, systems, and methods for plant propagation described herein include a processor (e.g., processor 33, 35, 37) electrically coupled to a fan or air compressor, a liquid valve, a first gas valve used to provide gas to the gas inlet of the replication capsule, a second gas valve used to provide gas to a bubble generator (that generates bubbles or nanobubbles in a liquid provided to the liquid inlet using the liquid valve), and the light source. In some cases, the processor (e.g., processor 33, 35, 37) instructs or causes the components described above to control the gas flows, liquid flow(s), gas mixture compositions, liquid mixture compositions, illumination parameters based on predefined parameters or in response to information or signals from a sensor.

In some embodiments, the devices, systems, and methods for plant propagation described herein include one or more sensors to measure environmental parameters or system process parameters, such as image sensors or cameras, temperature sensor(s), humidity sensor(s), vapor pressure deficit sensor(s), air exchange rate sensor(s), electrical conductivity sensor(s), pH sensor(s), carbon dioxide concentration sensor(s), oxygen concentration sensor(s), ethylene concentration sensor(s), nitrogen sensor(s), light intensity, light photoperiod, light spectrum sensor(s), liquid level sensor(s), gas flow meter(s), liquid flow meter(s), and microbial activity sensor(s). The sensors can provide information to a processor (e.g., processor 33, 35, 37) of a control system, and the processor (e.g., processor 33, 35, 37) can direct components of the device or system to actuate. For example, the processor (e.g., processor 33, 35, 37) can provide an instruction to a light, fan, air compressor, electronic liquid valve, or electronic gas valve.

In some embodiments, the devices, systems, and methods for plant propagation described herein include a functionally self-cleaning and self-balancing plant tissue culture propagation system which can enable cost-effective and localized vegetative plant propagation of a wide range of plant species with similar or higher propagation and rooting rates as compared to laboratory-based plant tissue culture micropropagation.

In some embodiments, the devices, systems, and methods for plant propagation described herein include a system-supported method of plant tissue culture propagation which eliminates high capital and operating costs associated with maintaining plant tissue cultures in an aseptic environment with sterile growing containers by employing mechanisms to automatically maintain balance between vegetatively propagating young plants and beneficial microbiota, and therefore eliminating the need for complex aseptic procedures and sterilization by a user.

In some embodiments, the devices, systems, and methods for plant propagation described herein include a temporary immersion of vegetative cuttings in clean, nutrient-rich, propagation-stimulating liquid within a non-sterile capsule housed under LED light fixtures.

In some embodiments, the devices, systems, and methods for plant propagation described herein include a system designed to achieve a balanced and dynamic environment which maintains favorable conditions for propagation within each capsule by maintaining any combination of beneficial microbiota, generated nanobubbles, gentle cleaning solutions such as polyoxyethylene sorbitan (e.g., Tween® 20) or hypochlorous acid, electrical conductivity, air exchange, concentrations of gases (e.g., CO₂, O₂, N₂, or ethylene gas), gas exchange rate, and the use of clean water and biochemical stimulants to temporarily immerse developing young plants within the replication capsule.

In some embodiments, the devices, systems, and methods for plant propagation described herein include a system designed to achieve higher transplant survival rates and faster acclimatization than traditional tissue culture within sterilized vessels. The improved survival rates can be due to environmental modulation during the rooting or hardening phase within the replication capsule, for example, due to adjustments made to gas exchange, temperature, and lighting intensity and spectrum in preparation for planting ex vitro. Additionally, exposure to beneficial microbiota within the replication capsule can impart hardiness benefits to the plantlets.

In some embodiments, the devices and systems for plant propagation described herein can be installed as a stand-alone system without specialized cleanroom or laboratory equipment or infrastructure and are suitable for onsite production at a facility by connecting the system to an electrical source and optionally a water source. For example, the system can be designed to be installed within an existing building, such as a warehouse.

In some embodiments, the system is designed to be installed within a shell such as a shipping container that is connected to electrical power and a water sources.

In some embodiments, the devices and systems for plant propagation described herein is designed to support production by enabling space-efficient, high-throughput, on-site vegetative propagation of thousands of young plants per square foot annually for agricultural, horticulture, or ecological production applications.

In some embodiments, the devices and systems for plant propagation described herein are designed to support scientific education whereby students can observe and assist in the propagation of a diverse range of ecologically and agriculturally important plants which are not produced from seed.

In some embodiments, the devices and systems for plant propagation described herein are designed to support research and development of new and improved protocols for vegetative propagation by testing multiple protocols and comparing their effectiveness. For example, algorithms, set points, acceptable ranges, and programs or protocols can be used to control the systems according to designed experiments. Developing plant tissue culture plantlets can be objectively assessed using a scoring system which uses numeric values related to different quality factors. For example, quality factors can be in the areas of morphology, presence of hyperhydricity, coloration, propagation rate, bacterial and fungal contamination level, survival rate, root development, and an overall score. Experimental environmental, nutritional, and/or microbiotic protocols can be designed, the systems can be used to carry out the experiments (e.g., using automation controlled by a processor). The results of using the experimental protocols can be assessed using the quality factors, and new environmental, nutritional, and microbiotic protocols can be recommended.

In some embodiments, the devices and systems for plant propagation described herein can include a control system with a processor (e.g., processor 33, 35, 37), and the processor can control components of the system to execute a program or protocol. In some cases, the program or protocol can be based on predefined parameters, and in some cases, the program or protocol can be based on predefined parameters and on feedback from one or more sensors. In some cases, the program or protocol can include changing one or more of nutrient liquids, beneficial microbiota liquids, cleansing agent liquids, light parameters, and environmental conditions in different stages that correspond to the different stages of development of a plantlet (establishment, replication, rooting, and acclimatization). In some cases, the program of protocol can additionally include cleaning cycles using a cleansing agent.

In some embodiments, the devices and systems for plant propagation described herein are modular and can be scaled by connecting multiple replication capsules to sources of liquid, gas, and light, using supporting equipment. For example, multiple replication capsules can be coupled to one or more centralized liquid sources, gas sources, and controls systems including a processor (or one or more processors). The replication capsules can be connected in series, parallel, or a combination of series and parallel configurations in order to efficiently scale the size and plantlet capacity of the systems. For example, large systems could be implemented using a plurality of the replication capsules described herein (e.g., from 2 to 20, from 10 to 100, or from 100 to 1000, or more than 1000 replication capsules) coupled to one or more centralized liquid sources, gas sources, and controls systems to suit the needs of a plant producer seeking to produce hundreds to thousands to millions of young vegetatively propagated plants annually.

In some embodiments, the devices and systems for plant propagation described herein include a set of transparent plant replication capsules, which are connected to liquid and gas inlets, and housed under an illumination source (e.g., an LED light fixture). A processor (e.g., processor 33, 35, 37) of a control system can execute an environmental protocol developed to suit the plant variety propagating therein. The protocol can include modulation of environmental conditions such as temperature, humidity, vapor pressure deficit, electrical conductivity, pH, carbon dioxide concentration, oxygen concentration, light intensity, light spectrum, photoperiod (i.e., the number of hours that a light is on per day), pre-mixed biochemical nutrient flood interval and volume schedule, and microbiota injection interval and volume schedule. The protocol can be a pre-programmed or can be a pre-programmed to include response to measurements from one or more sensors of the device or system. The one or more sensors to measure environmental parameters or system process parameters can include any of image sensors or cameras, temperature sensor(s), humidity sensor(s), vapor pressure deficit sensor(s), air exchange rate sensor(s), electrical conductivity sensor(s), pH sensor(s), carbon dioxide concentration sensor(s), oxygen concentration sensor(s), ethylene concentration sensor(s), nitrogen sensor(s), light intensity, light photoperiod, and light spectrum sensor(s), liquid level sensor(s), gas flow meter(s), liquid flow meter(s), and microbial activity sensor(s). The processor can instruct components of the device or system to execute the environmental protocol, for example. The processor can provide an instruction to a light, fan, air compressor, electronic valve, or air mixer.

In some embodiments, the devices and systems for plant propagation described herein can use a processor that can execute one or more algorithms to achieve one or more set points, or acceptable ranges. The processor may execute one or more programs or protocols to test biochemical, nutrient, and environmental experimental protocols and assess and/or adapt the experimental protocol based on measured effectiveness. For example, assessment can be performed by capturing and automatically grading photos of the young plants propagating therein. Photos can be graded on any of shape/morphology, presence of hyperhydricity, coloration, growth rate, bacterial and fungal contamination level, survival rate, root development. The processor, executing one or more operations, can instruct the system to modulate and detect reactions to any of biochemical and nutrient ratios, temperature, humidity, vapor pressure deficit, electrical conductivity, pH, carbon dioxide concentration, oxygen concentration, light intensity, light spectrum, photoperiod, nutrient flood interval and volume schedule, microbiota injection interval and volume schedule. For example, discrete quantities of different nutrients and/or biostimulants (e.g., micro-organisms, natural or artificial plant growth regulators, biofertilizers, or other materials used to promote plantlet growth) may be extracted from one or more source containers and mixed within the system to create a customized formula based on prior results or pre-programmed values. The algorithms, set points, acceptable ranges, and programs or protocols can also be adapted based on measurements from one or more sensors of the device or system. For example, one or more sensors can be used to measure environmental parameters. The environmental parameters can include any of temperature sensor(s), humidity sensor(s), vapor pressure deficit sensor(s), electrical conductivity sensor(s), pH sensor(s), carbon dioxide concentration sensor(s), oxygen concentration sensor(s), ethylene concentration sensor(s), nitrogen sensor(s), light intensity or spectrum sensor(s), liquid level sensor(s), gas flow meter(s), liquid flow meter(s), and microbial activity sensor(s). The data can then be analyzed to develop a new protocol based on pre-programmed information and/or information from sensor measurements, and the processor can instruct components of the device or system to execute the new protocol.

In some embodiments, the devices and systems for plant propagation described herein include a single-pass system where plantlets (e.g., vegetative cuttings) are harvested from a disease-free source plant, pre-treated with a mild cleansing solution, and inserted into the replication capsule. The plantlets can then be periodically submerged in clean, never-used liquid containing one or more biochemical nutrient formulas to induce propagation, root growth, and/or plantlet development. After the submersion timeframe has elapsed, the liquid can be drained into a waste reservoir or directly into a waste plumbing system of a building without being recirculated.

In some embodiments, the devices and systems for plant propagation described herein include a hybrid system with limited recirculation and outflow where plantlets (e.g., vegetative cuttings) are harvested from a disease-free source plant, pre-treated with a mild cleansing solution, and inserted into the replication capsule. The plantlets can then be periodically submerged in a liquid containing one or more biochemical nutrient formulas to induce propagation, root growth, and/or plantlet development. The liquid in the replication capsule can then be drained, collected in a reservoir, and recirculated back into the replication capsule. In some cases, the liquid can be recirculated a predefined number of cycles before the liquid drains into a waste reservoir or directly into a waste plumbing system of a building without additional recirculation cycles.

In some embodiments, the devices and systems for plant propagation described herein include a sanitized then recirculated system where plantlets (e.g., vegetative cuttings) are harvested from a disease-free source plant, pre-treated with a mild cleansing solution, and inserted into the replication capsule. The plantlets can then be periodically submerged in sanitized liquid containing one or more biochemical nutrient formulas to induce propagation, root growth, and/or plantlet development. The liquid in the replication capsule can then be drained, collected in a reservoir, and sanitized (e.g., using filter sterilization, UV-C light, chemical treatment, or heat) before being recirculated back into the replication capsule. In some cases, the liquid can be sanitized and recirculated a predefined number of cycles before the liquid drains into a waste reservoir or directly into a waste plumbing system of a building without additional recirculation cycles.

In some cases, the devices and systems for plant propagation described herein include a hybrid system with intermittent sanitation and recirculation. For example, plantlets (e.g., vegetative cuttings) are harvested from a disease-free source plant, pre-treated with a mild cleansing solution, and inserted into the replication capsule. The plantlets can then be periodically submerged in liquid containing one or more biochemical nutrient formulas to induce propagation, root growth, and/or plantlet development. The liquid in the replication capsule can then be drained, collected in a reservoir, recirculated back into the replication capsule. After a determined or predefined number of sanitation cycles, the liquid can be sanitized (e.g., using filter sterilization, UV-C light, chemical treatment, or heat), and then the sanitized liquid can be recirculated back into the replication capsule. In some cases, the liquid can be sanitized a determined number of times before the liquid drains into a waste reservoir or directly into a waste plumbing system of a building without additional recirculation cycles.

In some embodiments, the devices and systems for plant propagation described herein include a (1) single-pass system which exposes the plants to a liquid containing nutrients; (2) water with bubbles (e.g., nanobubbles) containing oxygen or another gas; and (3) a self-cleaning container achieved using liquid containing beneficial microbiota and liquids with cleansing agents.

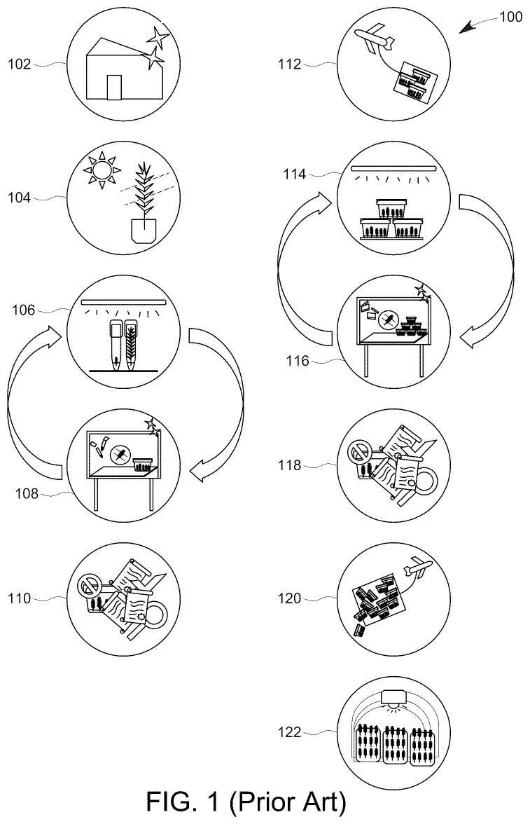

FIG. 1 shows a method 100 of a conventional process for culturing plants in vitro. Block 102 illustrates lab construction in a conventional process in order to maintain aseptic growing and processing conditions. Capital expenses, specialized infrastructure, laboratory equipment maintenance, and operating costs of a laboratory are all higher than other agricultural or horticultural facility types. Block 104 illustrates the labor-intensive process of harvesting explants from a healthy mother plant by extraction of a portion of the plant with adventitious growth potential. The extracted plant tissue is then heavily sanitized and dissected by the operator using an aseptic procedure, sometimes with the use of multiple sanitation and dissection steps and a microscope. Due to the negative effects of over-sanitation of small or microscopic sections of plant tissue, explants frequently become non-viable or die during or shortly after this step. Conversely, if the section of explant tissue is exposed to an insufficient level of sanitation in this step, bacteria, fungus, or other pathogens will spoil the culture, also resulting in non-viability or death of the explant.

Block 106 illustrates the development of the adventitious plant tissue into viable young plants with the support of laboratory infrastructure and a growth media in which multiple components are precisely measured, hydrated, tested for pH and electrical conductivity, then sterilized in advance and contained within sterile growth vessels which are either disposed of (creating abundant waste), or washed after each single use. Temperature and photoperiod may be controlled in this step by adjusting the ambient temperature or photoperiod, or with the additional investment in specialized chambers to segregate the sterile vessels. Lighting intensity and spectrum may be controlled by adjustable lights, but such lighting is typically cost-prohibitive. Nutrient levels may only be adjusted by preparing and sterilizing a new batch of semi-solid nutrient media, then performing the aseptic transfer process (described in block 108) before plants have matured.

Alternatively, if a laboratory is utilizing liquid culture media, nutrient levels may be adjusted by preparing and sterilizing a new batch of liquid nutrient media, disconnecting the undesired media vessel and attaching the new media vessel using an aseptic procedure, then disposing of or washing the undesired media vessel. In general, other environmental factors, such as levels of humidity, carbon dioxide, oxygen, and ethylene gas cannot be controlled within existing, available, cost-effective plant tissue culture vessels. During the development stage, any bacteria, fungal spores, or other pathogens which may have come in contact with the plant during the transfer process or the growth media during creation or storage may multiply, eventually resulting in non-viability or death of one or more plants contained within the contaminated vessel. As a countermeasure, some laboratories incorporate antibiotics and antifungals into growth medium. This countermeasure temporarily suppresses pathogenic growth but decreases propagation efficiency while enabling unfavorable mutation of young plants and resistant pathogens over time.

Block 108 illustrates the propagation of young plants after approximately one month in which plants are first inspected for visible pathogenic contamination before transfer to an aseptic workstation for processing. Processing may include removal of developed young plants from growth vessels, division of plants using surgical tools, and transfer onto new specialized growth media which is prepared and sterilized in advance and contained within sterile growth vessels. Vessels with new young plants are placed under lighting within the laboratory, and vessels which originated the young plants are either discarded or washed and sterilized.

Block 110 illustrates a portion of a conventional process often elected by commercial horticulture producers to minimize high domestic labor costs. In this step, a small number of young plant clones, typically between ten and fifty, are scheduled for export to a foreign country where the clones may be received by a tissue culture laboratory capable of producing a high number of clones, thousands to millions, at lower costs. Preparation for a shipment of young tissue culture plants requires negotiation with a receiving laboratory, negotiation with foreign Customs agents, filing of paperwork with foreign governments, scheduling of next-day airfreight with specialized temperature control, specialized processing of plants to meet foreign Customs criteria, on-site inspection from a state government official to obtain a phytosanitary certificate, specialized package labeling, specialized labeling of young plants, coordination of carrier to airfreight pickup location, verification of receipt, troubleshooting and re-shipping in the event of lost or damaged plants, and all associated shipping, labor, and overhead costs. Due to the high complexity and time required during this step, one or more skilled inventory managers and several knowledgeable laboratory employees are typically required to execute a successful shipment. Commercial horticulture facilities often prepare for multiple shipments each month to supply anticipated future demand.

Block 112 illustrates the shipment of young plant clones from a U.S.-based laboratory to a foreign production laboratory following shipment preparations. Plants are received by a coordinator at the production laboratory when each step of shipment preparation was executed properly, and the shipment was not randomly selected for additional inspection at a Customs agency. Additionally, shipment of plants outside of their country of origin incurs fluctuating freight costs, taxes, generates a significant carbon footprint, and has the potential of introducing pathogens contracted within the U.S.-based laboratory to ecosystems and production systems of the receiving laboratory.

Block 114 illustrates a similar step to block 106 in the receiving foreign laboratory where the adventitious plant tissue develops into viable young plants with the support of laboratory infrastructure and a growth media. Temperature and photoperiod may be controlled in this step by adjusting the ambient temperature or photoperiod, or with the additional investment in specialized chambers to segregate the sterile vessels. Similar to block 106, nutrient levels may only be adjusted by preparing and sterilizing a new batch of semi-solid nutrient media, then performing the aseptic transfer process (described in block 108) before plants have matured.

Block 116 illustrates a similar step to block 108 in the receiving foreign laboratory where the propagation of young plants occurs after approximately one month in which plants are first inspected for visible pathogenic contamination before transfer to an aseptic workstation for processing. Processing includes removal of developed young plants from growth vessels, division of plants using surgical tools, and transfer onto new specialized growth media which is prepared and sterilized in advance and contained within sterile growth vessels. Vessels with new young plants are placed under lighting within the laboratory. Vessels which originated the young plants are either discarded or washed and sterilized.

Block 118 illustrates a similar step to block 110 where the shipment of an ordered number of tissue culture plants with developed roots is coordinated for receipt by a U.S.-based greenhouse facility to fulfill customer demand.

Block 120 illustrates a similar step to block 112 where the shipment of tissue culture plants from a foreign production laboratory is shipped to and received by the U.S.-based greenhouse when steps of the shipment preparation were properly executed. The same additional risks apply, where shipment of plants to the U.S. from a non-U. S. laboratory incurs fluctuating freight costs, taxes, generates a significant carbon footprint, and has the potential of introducing pathogens contracted outside of the U.S. to ecosystems and production systems of the commercial greenhouse, seller, and end-user. Additionally, poor quality is a common result of shipments, where plants are shipped while still immature, infected with visible pathogens, incur stress from heat, cold, or darkness during transit, or otherwise deteriorate for unknown reasons.

Block 122 illustrates the planting of received tissue culture plants. Young plants in poor condition are either discarded or planted. Plants in poor condition which are planted may receive resource-intensive remediation treatments. Plants which arrive in poor condition may undergo additional condition tracking, documentation, requests to produce additional young plants for future orders, and often lead to additional negotiation with the foreign production laboratory and lost sales. After planting, young plants are developed into varying levels of maturity within a commercial greenhouse before distribution to a seller and final sale to an end-user. Ecological pathogens originating from a non-U.S.-based laboratory may infect adjacent organisms at a seller facility or on the property of the end-user.

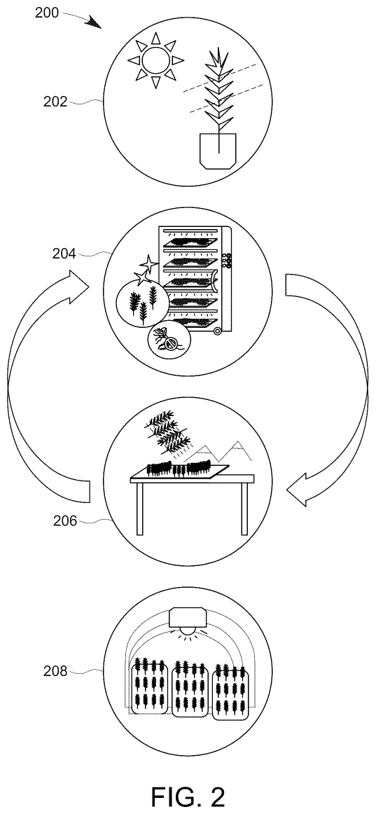

FIG. 2 shows an embodiment of a method 200 of culturing plants in vitro using the methods and devices described herein. As shown in FIG. 2, method 200 includes positioning one or more young plants or explants into a plant culture device, and receiving an input (e.g., at a processor of the device) of a type and/or a quantity of plant. The method 200 may optionally further include receiving, at the processor of the device, a scanned code for a specialized nutrient mix, and receiving an input of an acceptance or an override of a recommended growing protocol. The plants may develop inside the environmentally regulated plant tissue culture system, substantially free of contaminants. When desired growth is achieved, the plants may be divided for multiplication and various previous steps may be repeated, or the plants may be transferred to a greenhouse tray at a planting facility. This device may include various features to grow young plant clones without the traditional laboratory infrastructure and contamination control procedures (as shown in FIG. 1) and therefore dramatically reduces costs while improving plant quality and minimizing environmental risk of pathogens transferred from foreign territories.

As shown in FIG. 2, block 202 illustrates an embodiment of a process for harvesting explants from a healthy mother plant by extracting a portion of the plant with adventitious growth potential (e.g., an apical meristem, axillary bud, rhizome, dormant bud, basal shoot, or other portion). All plants have regions where new adventitious growth originates, which varies by plant species. The extracted plant tissue includes the removal of visible substrate and basic division without an aseptic procedure, microscope, or laboratory.

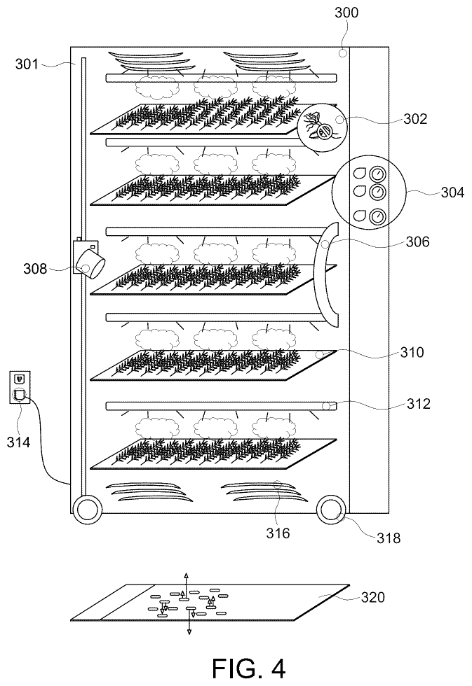

Block 204 illustrates the development of plants within an embodiment of a controlled and self-sanitizing environment of a plant tissue culture system, as shown in any of FIGS. 4-15. As described further herein, the devices, systems, and methods described herein include self-sanitizing, for example, using cleansing agents and nanobubble generators. Block 204 includes developing young plants from the adventitious plant tissue. Unlike the traditional process, plant development in the plant tissue culture system may occur in any location which is convenient for the user and uses a standard power outlet as opposed to laboratory infrastructure. Nutrient media may be prepared by stirring a premeasured packet of specialized nutrient mixture into distilled water and connecting the resulting mixture to the Plant Tissue Culture Growth Cabinet. Replacement or alteration of nutrient media may occur at any time using any of the methods described herein. The use of sterilization, aseptic procedures, and/or disposable containers is replaced by a self-sanitizing, highly reusable housing for plant development with the capacity for increased environmental control.

Environmental controls enable specific protocols and adjustment capability including one or more of: photoperiod, temperature, light intensity, light spectrum, humidity, carbon dioxide, oxygen, ethylene gas, or a combination thereof. Additionally, a rotation of dissimilar sanitizing agents and/or beneficial microorganisms may be applied at varying intervals to reduce or prevent plant disease and the unfavorable mutation of young plants and resistant pathogens over time. Multiple types of plants with similar nutritional and environmental requirements may be developed within the same plant tissue culture system due to the modular tray design, as shown in FIGS. 4-15.

As shown in FIG. 2, block 206 illustrates propagation of developed tissue culture plants. Young plants develop in the plant tissue culture system at approximately the same rate as in the traditional process, but less frequent handling may be used as plants may expand to fill a larger space. Propagation is achieved without the use of aseptic procedures or an aseptic environment, requiring far less training, workstation preparation, and precise handling, reducing overall labor cost. Once propagated, young plants can be returned to the plant tissue culture system where a sanitation cycle may eliminate any pathogens introduced during propagation.

Block 208 illustrates planting of young plants which have developed root tissue. After planting, young plants may be developed into varying levels of maturity within a commercial greenhouse before distribution to a seller and/or final sale to an end-user. By utilizing any of the plant tissue culture systems described herein, there may be limited, reduced, or no risk of foreign ecological pathogens infecting adjacent organisms at a seller facility or on the property of the end-user. Further, customized environmental protocols and control within the plant tissue culture system enable faster acclimatization to the greenhouse environment and reduce crop loss.

The systems, devices, and methods described herein enable highly specific protocols and adjustment capability including one or more of: photoperiod, temperature, light intensity, light spectrum, humidity, carbon dioxide, oxygen, ethylene gas, or a combination thereof. Nutrient mixtures may also be adjusted during plant development by simply mixing specialized nutrients into distilled water as opposed to an extended media creation process, as described above in correspondence with block 106 of FIG. 1. Additionally, a rotation of dissimilar sanitizing agents and/or beneficial microorganisms may be applied at varying intervals to prevent plant disease and the unfavorable mutation of young plants and resistant pathogens over time.

Further, unwanted financial and environmental costs associated with the traditional process, as shown in FIG. 1, include laboratory infrastructure, specialized HVAC and other recurring utilities, sterilization support equipment and maintenance, overhead and carbon costs of international shipping and administration (see, e.g., FIG. 1 at blocks 110, 112, 118, 120), lost product due to contamination and poor quality plants post-shipping, antibiotics to slow contamination rates which also stimulate mutation of resistant pathogens, high consumables and environmental costs of disposable containers, labor cost of media creation, and the overall cost and effort to maintain a clean laboratory environment.

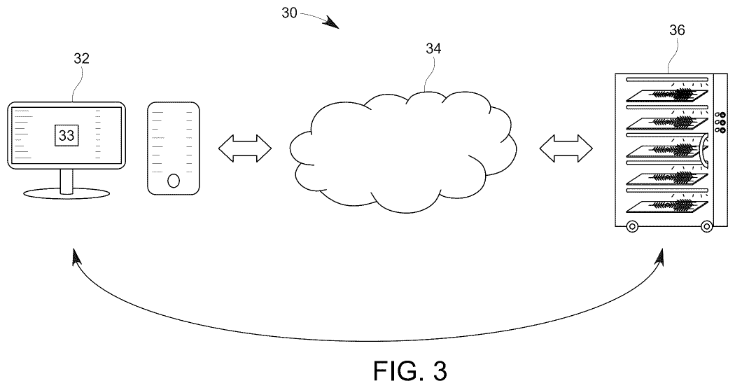

Turning to FIG. 3, which shows an embodiment of a method 30 of networked communication between a computing device 32, remote computing device 34, and a device 36 for growing one or more plants. FIG. 3 illustrates an exemplary, nonlimiting, flow of data which regulates the plant tissue culture system. For example, based on plant data inputs, one or more sensors transmit environmental and/or plant development signals, received at a plant culture device 36, to a remote computing device 34 (e.g., cloud, server, etc.) or computing device 32. A processor 37 of the plant tissue culture system 36 receives the transmitted signals, processes the signals to determine one or more environmental and/or plant development parameters, and outputs one or more signals, as described above, to modify the environment according to recommended protocols and/or user overrides. The plant tissue culture system may also be manually controlled remotely by a third-party.

Computing device 32 includes a processor 33 electrically coupled to a memory. The processor 33 may execute one or more operations stored in the memory. The operations may include receiving one or more inputs including, but not limited to, a plant type, a number of plants, and a desired quantity or output number of plants. Based on the one or more inputs, the computing device 32 may output a recommended protocol that may be accepted or overridden by a user. A remote computing device 34 including a processor 35 electrically coupled to a memory, may execute one or more operations stored in the memory. The remote computing device 34 may optionally receive from, and/or store, one or more protocols, recommendations, production forecasting data, plant development data, and/or user overrides. The remote computing device 34 may optionally transmit, to computing device 32, one or more protocols, recommendations, production forecasting data, plant development data, and/or user overrides. User-specific current and/or historic data may be accessed by a user of the system or device. In some embodiments, the data may be monitored and/or manually overridden upon request.

A plant culture device 36, for any devices of FIGS. 4-15, may regulate or control, using a processor 37 and a memory, a growth environment based on a protocol recommended to coincide with a particular nutrient pack, adjusting for one or more user overrides and/or transmitted plant development data, received from remote computing device 34 or computing device 32. Computing device 32 may be wirelessly connected to, or connected via a wired connection, to remote computing device 34. Computing device 32 may be wirelessly connected to, or connected via a wired connection, to plant culturing device 36. Remote computing device 34 may be wirelessly connected to, or connected via a wired connection, to plant culturing device 36. Any wireless protocol may be used, for example a local area network (LAN), Bluetooth, Zigbee, LoRaWAN, Sigfox, LTE, and the like. Each device 32, 34, 36 may include an antenna to receive and/or transmit data to any of another device 32, 34, 36.

Turning now to various embodiments of plant culture devices. For example, FIG. 4 illustrates a perspective cross-sectional view of an embodiment of a plant tissue culture system. Nutrient reservoir 300 may be a refillable liquid plant nutrient media canister that is in fluid communication with or coupled to a housing 301 of the plant tissue culture system. Liquid contained in nutrient reservoir 300 may be aerosolized for uptake by plants in housing 301. The nutrient media may be generated (manually or automatically) by dissolving a predefined quantity of specialized nutrient mixture into distilled water and causing the nutrient reservoir 300 to be put into fluid communication with the plant tissue culture system. A nutrient dispersion schedule may be calibrated to achieve a desired or an optimum contact time with plant tissue while conserving consumable resources.

Sanitization reservoir 302 may be a refillable sanitizing agent canister(s) in fluid communication with or coupled to the plant tissue culture system. Sanitizing agents may be rotated to avoid mutation of resistant pathogens and dispensed via aerosolized particle at a predefined interval to preserve plant health. In some embodiments, a plant tissue culture system and/or one or more components (e.g., shelving, fixtures, etc.) in the plant tissue culture system may include an antimicrobial coating to increase sanitation. In some embodiments, a plant tissue culture system may include a probiotic reservoir for introducing probiotics to one or more plant species or cultivars in the plant tissue culture system. In some embodiments, the plant tissue culture system may include a light source 312, for example an ultraviolet radiation light source to increase sanitation within the housing of the plant tissue culture system.

Interface 304 may include a series of input elements (e.g., buttons, switches, touch screens, dials, etc.) to control and/or monitor one or more of: light intensity, light spectrum, photoperiod, temperature, humidity, carbon dioxide, oxygen, and other gasses including ethylene. The plant tissue culture system may include an embedded hardware processor for receiving inputs from the input elements and activating one or more motors for releasing medium or sanitizing agents from a reservoir, activating a light source, activating a heat source or cooling element (e.g., Peltier, thermoelectric cooler, etc.), activating a gas source, activating a sensor to read a condition in the plant tissue culture system, etc. Alternatively, or additionally (as shown in FIG. 3), the plant tissue culture system 36 may be communicatively coupled (e.g., via a wired connection or wirelessly) to a remote computing device 32, 34 (e.g., mobile computing device, server, workstation, laptop, etc.) that includes a hardware processor for processing as above. The processor(s) may be coupled to memory having instructions stored thereon detailing one or more growth profiles, for example tied to a particular plant species. The growth profiles may be displayed and/or adjusted from the remote computing device 32, 34 and/or locally at the plant tissue culture system 36. The processor(s) can receive inputs, for example detailing plant growth requirements, sensor inputs (e.g., from sensors in the plant tissue culture system that are communicatively coupled to the processor(s)), and/or environmental and/or plant development data. As such, the processor(s) may receive these inputs and output a modified environment in the plant tissue culture system by modifying any of the parameters identified elsewhere herein. The outputs from the processor(s) may be based on recommended protocols and/or subject to user overrides. In some embodiments, the plant tissue culture system may also be remotely and/or manually controlled by a third-party operator, as shown in FIG. 3.

Handles 306 enable universal accessibility and/or locking, as new plant varieties are considered intellectual property of the plant breeder.

Sensor(s) 308 may be integrated with the plant tissue culture system, embedded on the plant tissue culture system, or otherwise coupled to the plant tissue culture system. For example, the sensors 308 can provide remote monitoring and/or automatic feedback for protocol improvement. Exemplary sensors include, but are not limited to: image sensors (e.g., charge-coupled device), temperature sensor, carbon dioxide sensor, humidity sensor, oxygen sensor, ethylene sensor, light sensor, etc.

Support 310 may be a removable tray which houses developing young plants, designed for ergonomic handling. Light source 312 can include spectrum and/or intensity-adjustable, moisture-proof LED lighting, to promote proper plant growth and environmental adjustment. Power source 314 for the plant tissue culture system can be a wall outlet, solar panels, battery, etc. such that plants can be propagated in any location. Vents 316 allow for safe gas exchange to regulate a growth environment and reduce acclimatization time, with increased efficiency due to natural convection. Mobility element(s) 318 can be industrial casters, wheels, or the like which allow for workflow adaptation, unlike most commercial greenhouse equipment. Modular trays 320 allow for production flexibility, gas exchange, contact time with nutrients and sanitation, and/or natural convection.

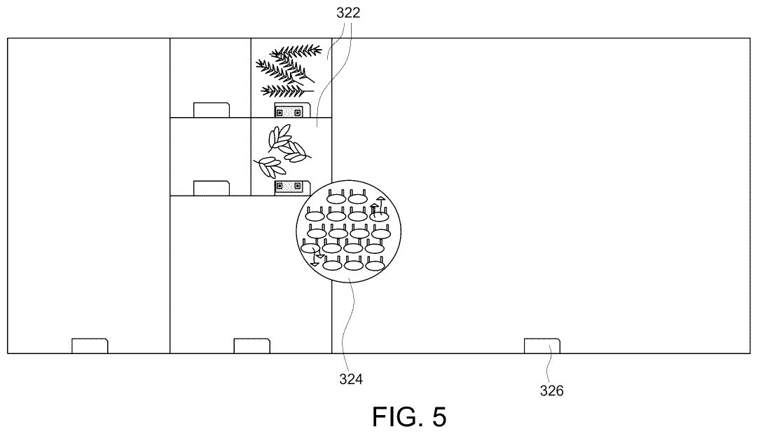

FIG. 5 illustrates an embodiment of a modular growth tray design. This design enables unique plant varieties with varying production demand and similar nutritional and environmental requirements to develop on independent schedules while maintaining batch integrity. As shown by element 322, which illustrates a top-down view of the modular tray inserts, multiple varieties are separated. For example, strawberry (Fragaria×ananassa), raspberry (Rubus idaeus), or blueberry (Vaccinium caesariense) plantlets can be separated as shown in FIG. 5. This separation accommodates varying production demands inside the plant tissue culture system. As demand increases and a plant order progresses, forecasted space may increase, and the user may select a larger tray, conserving space early in the production process.

Inserts 324 may include grooves for holding a tray in place. There may also be surrounding openings (surrounding the grooves) for heat circulation, gas exchange, and liquid exchange. Openings are small enough to prevent exchange of plant material between levels.

Alternatively, in some embodiments, one or more ventilated baskets positionable on an insert (e.g., shelf, grate, etc.) in the housing may be utilized to contain one or more plants.

Labels 326 (e.g., QR code, barcode, etc.) can be included on the tray inserts or elsewhere on the tray. Labels 326 can include plant and batch information throughout order progression. More of these labels may be created as an order increases in size to fill multiple shelves and contains the history of production and protocol data of that batch for improved future protocol development.

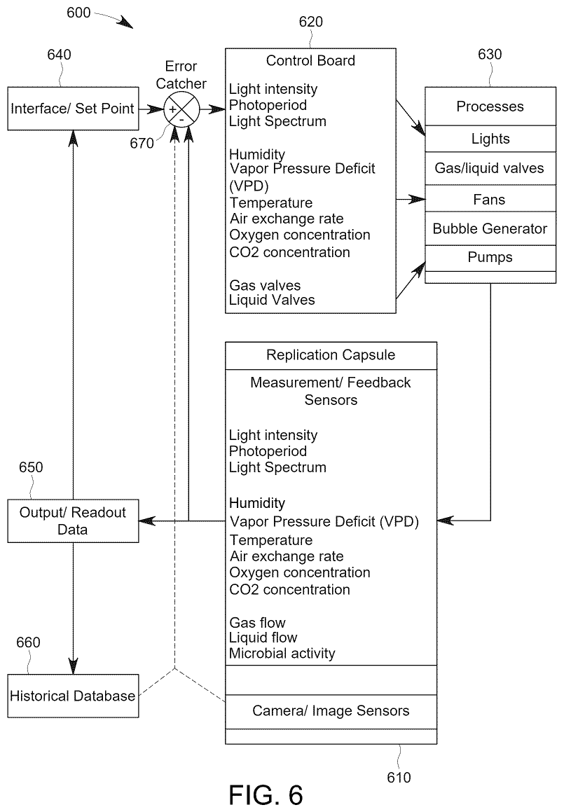

FIG. 6 shows a block diagram of an example system 600 for plant propagation, in accordance with some embodiments. The system 600 includes at least one replication capsule 610, control board 620, processes 630, input interface 640, output device 650, and database 660. A set point or program can be entered using the input interface 640. A processor of the control board 620 can use the set point or program to control a process 630, such as activating or deactivating a light source, opening or closing a gas valve, activating or deactivating a fan, opening or closing a liquid valve, and/or activating or deactivating a pump. The input interface 640/set point contains a set point, acceptable range, protocol, or program for each parameter that is controlled using the processor of control board 620.

The environment in the replication capsule 610 can be monitored using one or more sensors, such as temperature sensor(s), humidity sensor(s), vapor pressure deficit sensor(s), air exchange rate sensor(s), electrical conductivity sensor(s), pH sensor(s), carbon dioxide concentration sensor(s), oxygen concentration sensor(s), ethylene concentration sensor(s), nitrogen sensor(s), light intensity, light photoperiod, and light spectrum sensor(s), liquid level sensor(s), gas flow meter(s), liquid flow meter(s), and microbial activity sensor(s). Another type of sensor that can be included in replication capsule 610 is a camera or image sensor, in some cases. The output from the sensor(s) can be sent to the output device 650 and/or to database 660.

In some cases, the information from the sensor(s) can be used by the processor of the control board 620 to control processes 630. For example, a microbial activity sensor in replication capsule 610 can be used to detect the presence of a detrimental bacteria or biofilm, and the processor can receive and process the information from the microbial activity sensor to determine if a valve or series of valves should be opened to cause a liquid containing beneficial microbiota, or a liquid containing a cleansing agent, to flood the replication capsule 610.

The processor of control board 620 can execute an algorithm to determine how to control the processes 630 based on information from one or more sensors. In some cases, the algorithm can include ranges or thresholds for each sensor, where a particular process set point is specified for each range or threshold of each sensor. In some cases, the algorithm can receive as an input information from one or more sensors to control one or more processes. In some cases, the processor of control board 620 can use information from one or more sensors to trigger a series of actions for the processes 630. For example, a microbial activity sensor in replication capsule 610 can be used to detect the presence of a detrimental bacteria or biofilm, and the processor can use the information from the microbial activity sensor to determine if a cleaning protocol should be initiated. The cleaning protocol can include opening a valve or series of valves to cause a liquid containing a cleansing agent to flood the replication capsule 610. The cleaning agent may be drained (e.g., by opening one or more fluid output valves), and a valve or series of valves may be opened to cause a liquid containing beneficial microbiota to flood the replication capsule 610. Error detector 670 can be used by the processor of control board 620 to check whether a current process is within a set point, and output to the processor an adjustment to the set point or program as needed.

The processor of control board 620 can execute an algorithm to determine a vapor pressure deficit for a plant or a plurality of plants in a capsule or cabinet. The vapor pressure deficit may use pressure to measure the difference between saturation and the current amount of moisture in the air and is used to indicate how much moisture is being absorbed compared to generated by the plants within the system. For example, the leaf saturated vapor pressure (SVP) may be the same as the air SVP, but a temperature of the leaf may be cooler than that of the air (e.g., about 1° C. to about 3° C. or about 2° F. to about 5° F. cooler), leading to a vapor pressure deficit. An infrared thermometer may be used to measure a leaf temperature.

The processor of control board 620 can cause an activation of a liquid valve, which can cause a flow of liquid to be provided, for example to a liquid mixing tank, or to a container or capsule containing plantlets. A liquid tank can contain a volume of liquid and a tube can couple the liquid tank to liquid mixing tank, or a liquid inlet of a container or capsule containing the plantlets. In some embodiments, the liquid tank can be elevated with respect to the liquid mixing tank or liquid inlet causing the liquid to be under pressure in the tube. A valve between the liquid tank and the liquid mixing tank or liquid inlet can be used to control the flow of the liquid through the tube. For example, a robotically actuated pinch valve can be placed around a rubber tube coupling the liquid tank to the liquid inlet, and the pinch valve be electronically coupled to the processor so that the processor can control the flow of liquid through the tube. In some embodiments, a valve can be used to control the flow of liquid to be either on or off. In some embodiments, a valve can be used to continuously or discretely control a flow rate of liquid through the tube.

In some embodiments, the processor of control board 620 can cause an activation of a liquid spray. For example, activation of the liquid spray may be based on or determined using one or more of: a proof water pressure (e.g., less than about 0.8 MPa), a water flow range: (e.g., about 0.3 L/min to about 6 L/min), a voltage range (e.g., about 5 V to about 12 V), and/or an operating current (e.g., about 15 mA or about DC 5V). Liquid spray activation may further include a capacitive liquid detection.

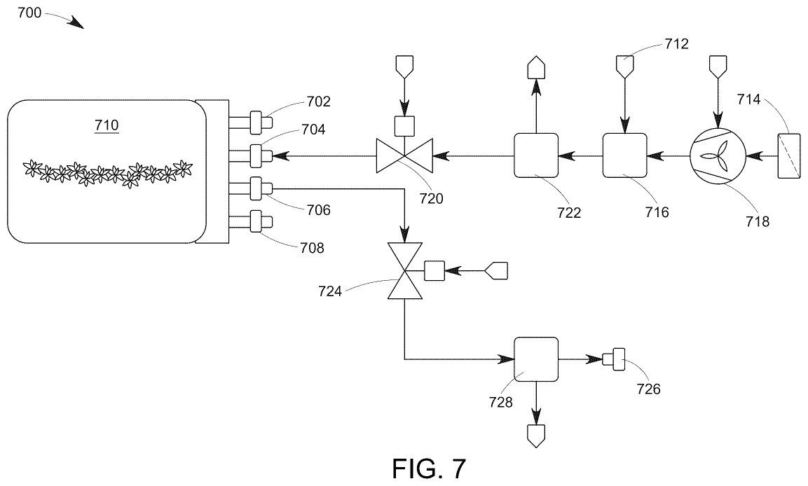

FIG. 7 shows a schematic example of a system 700 for plant propagation, in accordance with some embodiments. The replication capsule 710 may include a gas inlet 704, a gas outlet 706, a liquid inlet 702, and/or a liquid outlet 708. System 700 shows an example of a system to provide gas from a gas tank 712 to the replication capsule 710. Air may enter the system through an air filter 714 and may be pushed into a gas mixer 716 using an air compressor 718. Gas from the gas tank 712 may be mixed with air from the air compressor 718, and processor can cause the valve 720 to open, thereby allowing the gas mixture to enter the replication capsule 710 through the gas inlet 704. In this example, a gas sensor 722 may be positioned between the gas mixer 716 and the replication capsule 710, which can measure a concentration of a gas (e.g., carbon dioxide) in the gas mixture before it is allowed to enter the replication capsule 710. The processor can also control the gas valve 724 coupled to the gas outlet 706 to allow gas in the replication capsule 710 to exit through the gas outlet 706 and through a vent 726 with an optional air filter. In this example, a gas sensor 728 is between the gas outlet 706 and the vent 726, which can measure a concentration of a gas (e.g., carbon dioxide) after exiting the replication capsule 710. For example, a difference in carbon dioxide concentration entering the replication capsule 710 and exiting the replication capsule 710 can be used as an indicator of an amount of plantlet growth.

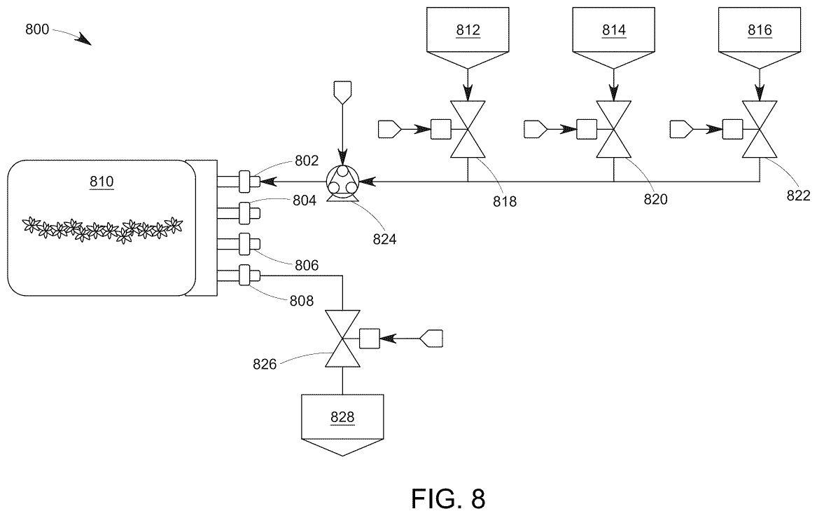

FIG. 8 shows a schematic example of a system 800 for plant propagation, in accordance with some embodiments. The replication capsule 810 may include a gas inlet 804, a gas outlet 806, a liquid inlet 802, and/or a liquid outlet 808. System 800 shows an example of a system to provide liquids from one or more liquid tanks to the replication capsule 810. For example, there may be a nutrient tank 812, an anti-microbial tank 814, and/or a probiotic tank 816. Although three tanks are shown, one of skill in the art will appreciate that there could be a fewer or greater number of tanks. A processor can control individual liquid valves, for example valve 818, valve 820, valve 822, coupled to the liquid tanks (e.g., tank 812, tank 814, tank 816) and a liquid pump 824 to deliver different liquids to the replication capsule 810 through the liquid inlet 802. In some cases, one or more liquid flow sensors (not shown) can be used to determine an amount of liquid delivered to the replication capsule 810 from the different liquid tanks 812, 814, 816. Liquid tanks described herein can include any vessel that can hold liquid, for example, a vat or container with a liquid outlet, or a cartridge with a liquid outlet that interfaces with a socket for the cartridge containing a liquid inlet. The liquid tank can be coupled to a liquid inlet using conduits, and gaskets, valves and other components to deliver predefined or predetermined quantities of liquid and to reduce or prevent leaks. The processor can also control the liquid valve 826 coupled to the liquid outlet 808 to allow gas in the replication capsule 810 to exit through the liquid outlet 808 and into a collection tank 828. This example shows a system 800 where the liquid is not recirculated, and fresh liquids are delivered to the replication capsule 810 and then drained into the collection tank 828. In other cases, the collection tank can be coupled to the liquid inlet 802 using a recirculation system (not shown).

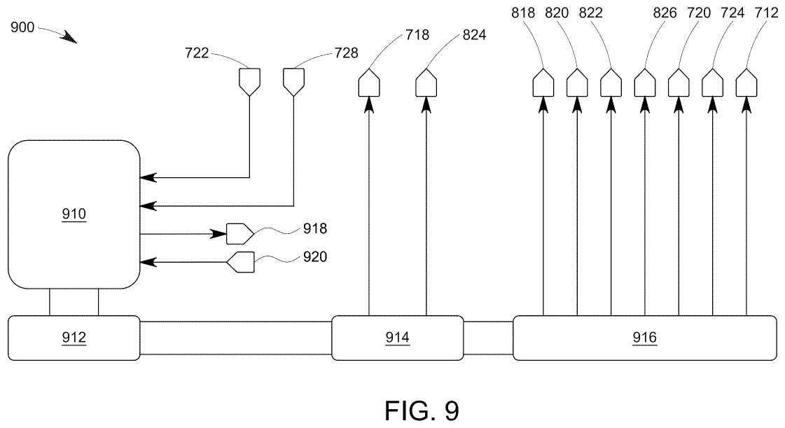

FIG. 9 shows a schematic example of a system 900 for plant propagation, in accordance with some embodiments. System 900 includes processor 910 electrically coupled to a first control board 912 (e.g., Hardware Attached on Top (HAT)) for a fan or air compressor, a second control board 914 (e.g., motor HAT) for a liquid pump, and a third control board 916 (e.g., driver HAT) for gas valves and liquid valves. For example, processor 910 of system 900 can be used to control the air compressor 718, gas valves 720, 724, and CO2 dosing of system 700 in FIG. 7, and the liquid pump 824 and the liquid valves 818, 820, 822, 826 of system 800 in FIG. 8. Further for example, processor 910 may receive signal inputs from sensors 722, 728, process the signals, and output one or more indications, recommendations, processes, or parameters. In some embodiments, processor 910 may output to a display 918 and/or receive one or more inputs via input element 920.

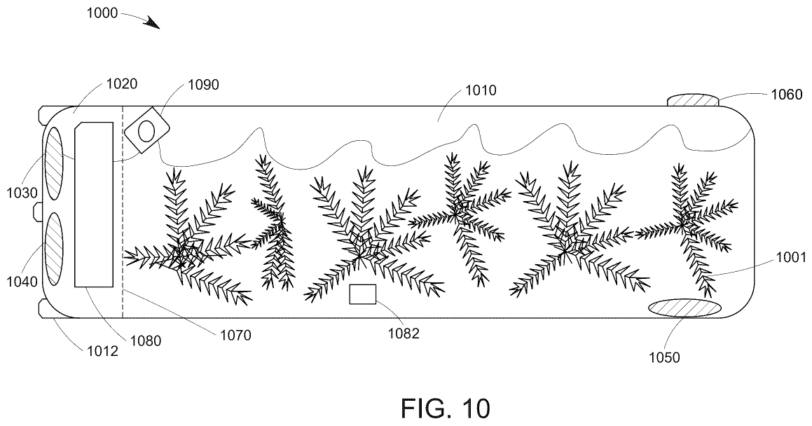

FIG. 10 shows an example of a replication capsule 1000 for plant propagation, in accordance with some embodiments. Replication capsule 1000 may include a capsule body 1010, mounting hardware 1012, capsule cap 1020, liquid inlet 1030, gas inlet 1040, liquid outlet 1050, gas outlet 1060, a membrane 1070, sensors 1080 and 1082, and an image sensor 1090. Replicating plantlets 1001 are also shown in an internal volume defined by the replication capsule 1000. At least a portion of capsule body 1010 is transmissive to light. For example, capsule body 1010 can be made partially or entirely of a translucent material, for example a plastic or a polymer. The capsule cap 1020 can be detached from capsule body 1010, for example, to insert or remove plantlets. The membrane 1070 can allow gas and liquid exchange across the membrane 1070 and can prevent plantlets 1001 from contacting the liquid inlet 1030, the gas inlet 1040, or the sensors 1080. Membrane 1070 can also prevent plantlets 1001 from falling out when the capsule cap 1020 is removed from the capsule body 1010.

Sensors 1080 and 1082 can each include one or more sensors, such as temperature sensor(s), humidity sensor(s), vapor pressure deficit sensor(s), air exchange rate sensor(s), electrical conductivity sensor(s), pH sensor(s), carbon dioxide concentration sensor(s), oxygen concentration sensor(s), ethylene concentration sensor(s), nitrogen sensor(s), light intensity, light photoperiod, and light spectrum sensor(s), liquid level sensor(s), gas flow meter(s), liquid flow meter(s), and microbial activity sensor(s). For example, sensor 1082 can include a microbial activity sensor which can provide information about the presence of microbes or biofilms in the replication capsule in the vicinity of the plantlets 1001. Sensor 1080, for example, can include carbon dioxide sensor(s), which can provide information (e.g., concentration) about the carbon dioxide content in the air in the replication capsule and be protected from the plantlets by the membrane 1070. The sensors 1080 and 1082 and the image sensor 1090 can be coupled to a processor (e.g., processor 910 in system 900 in FIG. 9) to provide information about the environment in the replication capsule 1000 and/or about liquids and/or gases entering and/or leaving the replication capsule 1000. The gas inlet 1030 can be coupled to one or more gas tanks, for example, as shown in system 700 in FIG. 7. The liquid inlet 1030 can be coupled to one or more liquid tanks, for example, as shown in system 800 in FIG. 8.

Liquid can be delivered to replication capsule 1000 either using a flood or a spray delivery mechanism. In the case of a flood delivery mechanism, a conduit carrying the liquid from the liquid tank(s) may be coupled to the liquid inlet 1030, and when the processor instructs a liquid valve to open and/or a liquid pump to turn on, then the liquid fills the bottom of the replication capsule with a volume of the liquid (e.g., from about 0.1 L to about 2 L, or from about 0.1 L to about 1 L, or from about 0.2 L to about 0.5 L). After a period or a duration of time, the volume of liquid may be drained. In the case of a spray delivery system, an atomizer or a spray nozzle can be coupled to the liquid inlet 1030, and when the processor instructs a liquid valve to open and/or a liquid pump to turn on, then the liquid may be sprayed in the interior volume defined by the replication capsule. In the case of the spray delivery cases, there can be continuous draining of the liquid, or a combination system could include a spray delivery system controlled to deliver a volume of the liquid (e.g., from about 0.1 L to about 2 L, or from about 0.1 L to about 1 L, or from about 0.2 L to about 0.5 L) over a certain length of time without draining such that the plantlets are immersed in a volume of the liquid that collects at the bottom of the replication capsule. In this case, after a period or a duration of time, the pool of liquid may be drained.

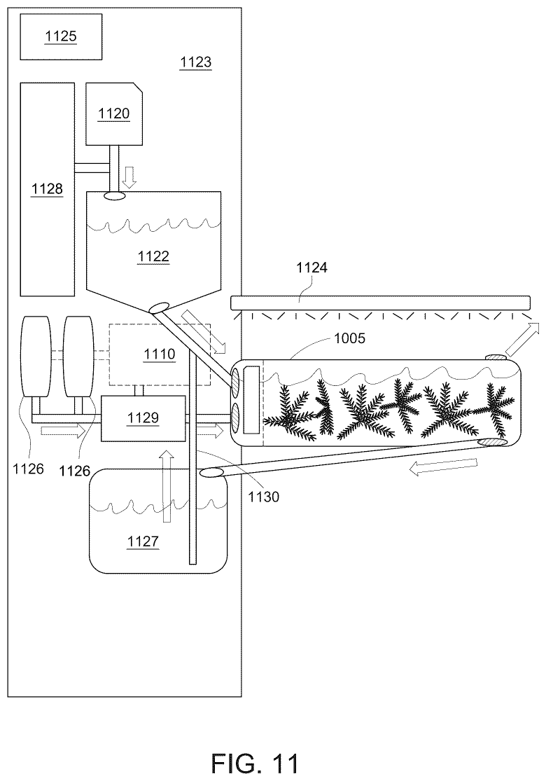

FIGS. 11-13 show schematic examples of systems for plant propagation, in accordance with some embodiments. The systems shown in FIGS. 11-13 each contain similar components that are arranged differently and/or coupled together differently in the different embodiment shown in the figures. These systems each include one or more replication capsules 1005 similar to replication capsule 1000 in FIG. 10. Light sources 1124 (e.g., LED lights, incandescent lights, or fluorescent lights) are arranged to illuminate plantlets within the replication capsules 1005. There are one or more gas tanks 1126, and one or more liquid tanks 1120 coupled to the gas inlet(s) and liquid inlet(s) of the one or more replication capsules 1005. The systems in FIGS. 11-13 also include a bubble generator 1110 positioned between a liquid tank 1120 and the liquid inlet(s) of the replication capsule(s) 1005. In some cases, bubble generator 1110 is a nanobubble generator that generates small bubbles with average diameters less than a micron in diameter. The bubble generators 1110 in these systems are also coupled to one or more of the gas tanks 1126, which may provide the gas that forms the bubbles in the liquid that is delivered to the replication capsule(s) 1005. These systems also show an example where a liquid mixing tank 1122 may be used to mix water, for example water prepared using reverse osmosis water filtration 1128, with a liquid from a liquid tank 1120. These systems also show examples where different gases from different gas tanks are mixed (optionally with air) using a gas mixer 1129. The gas mixer 1129 can mix gases such as carbon dioxide, oxygen and/or ethylene with air to provide air with excess carbon dioxide (e.g., air with carbon dioxide concentration higher than about 200 ppm to 500 ppm, or higher than 500 ppm), or air with excess oxygen (e.g., air with oxygen concentration higher than about 21%) to the replication capsule 1005 and/or bubble generator 1110. For example, one of the gas tanks 1126 can contain air with about 200 ppm to about 500 ppm of carbon dioxide, and another gas tank 1126 can contain oxygen to mix with air, or oxygen premixed with air. The liquid can drain from a liquid outlet to a collection tank 1127. These systems also include a computing device 1125, which can include a processor to control the system, for example, as described with respect to systems 700, 800 and 900 in FIGS. 7-9. The components of these systems can be housed in an enclosure or can be mounted to a board or wall 1123. The arrows indicate the direction of gas flow or liquid flow, which can be controlled using the processor, as described herein.

The systems shown in FIGS. 11-14 can include a plurality of gas tanks 1126, such as two, three, four, five, six, or more than six gas tanks 1126. Gas tanks 1126 can hold the same or different gas mixtures from one another. For example, Gas tanks 1126 can include air, oxygen, carbon dioxide, ethylene, nitrogen, or mixtures thereof. The gas tanks 1126 can be coupled to the gas mixer, which can provide mixed gas to the bubble generator 1110 or the replication capsule 1005. In some embodiments, the systems shown in FIGS. 11-14 can include a plurality of valves coupled to a processor, where the valves are positioned between the gas tanks 1126 and the gas mixer 1129, and between the gas mixer 1129 and the replication capsule 1005. The processor can control the valves to control the to control the mix of gases provided to the replication capsule 1005, and the mix of gases provided to the bubble generator 1110 to generate bubbles (e.g., nanobubbles) containing the gas mixture. As described herein, the gas environment in the replication capsule 1005 can be controlled separately from the gas used to generate the bubbles. For example, air with excess carbon dioxide can be provided to the environment for the plantlet leaves to absorb, and air with excess oxygen can be provided to the bubbles for the plantlet roots to absorb. Other gases such as ethylene can also be beneficial to the plantlets. Gases such as nitrogen can be beneficial for the environment, for example, to influence the growth of the harmful or beneficial microbiota in the replication capsule 1005.

FIG. 11 shows a schematic example of a system for plant propagation, including a replication capsule 1005, a liquid tank 1120, and two gas tanks 1126. In some cases, fresh liquids can be delivered from the liquid mixing tank 1122 to the replication capsule 1005 and drain without being recirculated or reused. In other cases, the liquids from the replication capsule 1005 can be collected and recirculated, for example using optional recirculation system 1130. The collection tank 1127 can include a sanitizing system (e.g., using UV light, filters, or heat) to clean the liquid before recirculation. The system in FIG. 11 includes one liquid tank, but in other embodiments there can be more than one liquid tank 1120. In such cases, multiple liquid tanks can feed into a single mixing tank, or each liquid tank can have its own water supply and mixing tank. In such cases, there can also be multiple bubble generators between each mixing tank and the liquid inlet of the replication capsule 1005.

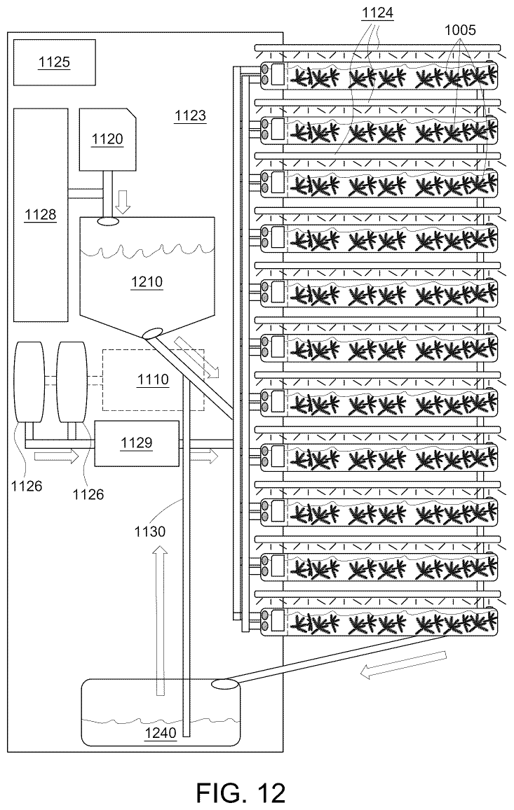

FIG. 12 shows a schematic example of a system for plant propagation, including multiple replication capsules 1005 coupled to one liquid tank 1120, and two gas tanks 1126. There is a plurality of replication capsules 1005 in this example (e.g., more than about 10), coupled together in parallel, meaning that liquid is delivered from the mixing tank directly to each replication capsule 1005 (in parallel) and then the waste is collected. In some cases, fresh liquids can be delivered from the liquid mixing tank 1210 to the replication capsules 1005 and then drain without being recirculated or reused. In other cases, the liquids from the replication capsule 1005 can be collected and then recirculated, for example using optional recirculation system 1130. The collection tank 1240 can include a sanitizing system (e.g., using UV light, filters, or heat) in this case to reduce the risk of cross-contamination between the replication capsules 1005 due to the recirculated liquids. The system in FIG. 11 includes one liquid tank 1120, but in other embodiments there can be more than one liquid tank 1120. In such cases, multiple liquid tanks can feed into a single mixing tank, or each liquid tank can have its own water supply and mixing tank. In such cases, there can also be multiple bubble generators (e.g., nanobubble generators) between each mixing tank and the liquid inlets of the replication capsules 1005. In cases where there are multiple mixing tanks, then each mixing tank can supply multiple replication capsules 1005 in parallel as shown in FIG. 12. In some cases, a series of valves (not shown) between the liquid mixing tank 1210 and the plurality of replication capsules 1005 can be used to deliver different liquid mixtures to different replication capsules 1005. Such a system of valves can be coupled to the processor, which can control the valves to deliver different liquid mixtures to different replication capsules 1005 over time, for example, in response to information from sensors in the different replication capsules 1005, or in order to perform experiments to develop new plant replication protocols.

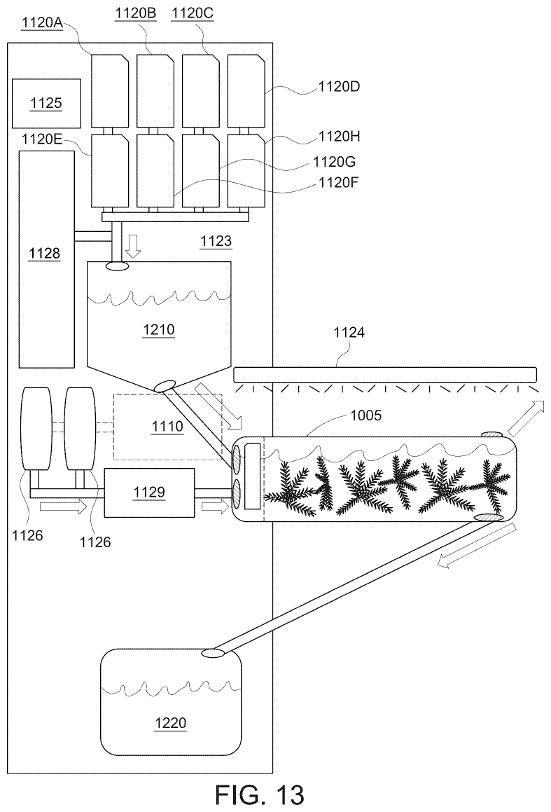

FIG. 13 shows a schematic example of a system for plant propagation, including a replication capsule 1005 where fresh liquids are introduced from multiple liquid tanks and then collected without being recirculated, in accordance with some embodiments. The system in FIG. 13 includes a plurality of liquid tanks 1120A, 1120B, 1120C, 1120D, 1120E, 1120F, 1120G, 1120H coupled to a mixing tank 1210. A water source is also coupled to the mixing tank. A series of valves (not shown) similar to the valves shown in FIG. 8 can be controlled using a processor to deliver specified volumes of liquid from the liquid tanks 1120 to the mixing tank 1210. In some cases, one or more liquid pumps (not shown) similar to the liquid pump in FIG. 8 can be used to deliver the liquids from the liquid tanks 1120 to the mixing tank 1210. In some cases, the force of gravity can be used along with the valves to deliver the liquids from the liquid tanks 1120 to the mixing tank 1210. For example, the liquid tank(s) can be elevated with respect to the replication capsule, such that when valves are opened, the force of gravity can deliver the liquid from the tank to the replication capsule. The different liquid tanks 1120 can contain liquids with different nutrients, combinations of nutrients, beneficial microbiota, or combination of nutrients and beneficial microbiota. In some cases, the liquids in one or more of the liquid tanks 1120 can contain liquids with cleansing agents used to clean the plantlets and/or the interior of the replication capsule 1005. The bubble generator 1110 can advantageously generate bubbles in any of the liquids after they are mixed and optionally diluted in the mixing tank. In this example there is no recirculation, however, a recirculation system similar to recirculation system 1130 in FIGS. 11 and 12 could be added to the system in FIG. 13.

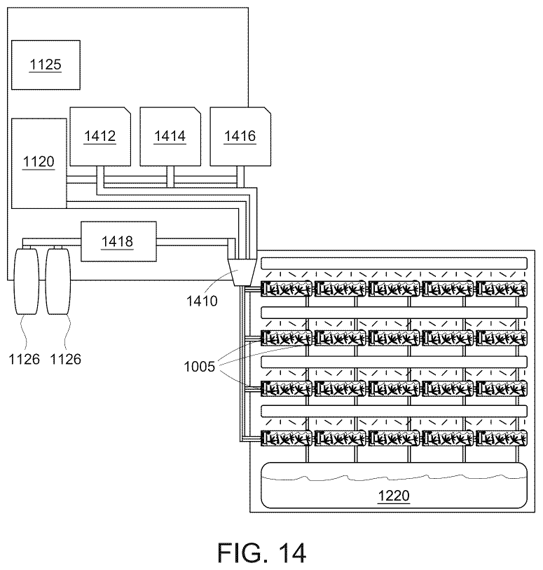

FIG. 14 shows a schematic example of a system for plant propagation, including multiple replication capsules 1005, multiple liquid tanks, and two gas tanks. The components of the system in FIG. 14 are similar to those in FIGS. 11-13 described above. The system in FIG. 14 includes three liquid tanks, one containing a nutrient mixture 1412, one containing beneficial microbiota 1414, and one containing a cleansing agent 1416. The system in this example can include a mixing tank, bubble generator (e.g., nanobubble generator), and/or gas mixer 1410 with components similar to the mixing tank shown in the systems in FIGS. 11-13, a bubble generator similar to the bubble generator 1110 shown in the systems in FIGS. 11-13, and a gas mixer similar to the gas mixer 1418 shown in the systems in FIGS. 11-13. The system in FIG. 14 can include a processor that can control valves, fans or air compressors, and liquid pumps, as described above with respect to the systems in FIGS. 7-13. For example, the processor of the computing device 1125 in the system in FIG. 14 can control the system to deliver nutrients, beneficial microbiota, and cleaning agents using a series of valves, to execute a program for plantlets within the replication capsules 1005.

The system in FIG. 14 includes a plurality of replication capsules 1005 (e.g., about 20) coupled in a series-parallel configuration. In other words, there is a single source of gases and a single source of liquids that feeds the plurality of replication capsules 1005, where there are five replication capsules 1005 coupled in series, and four of these groups of replication capsules 1005 coupled in parallel. In such an arrangement, fresh gases and liquids are directly delivered to four of the replication capsules 1005 from the gas tanks 1126 and liquid tanks 1120, and then liquid is delivered from one replication capsule 1005 to the others coupled to it in series. In the example shown, each replication capsule 1005 has its own drain line coupling the replication capsule 1005 to the collection tank 1220, which can be advantageous to avoid cross-contamination between the replication capsules 1005. In this example there is no recirculation, however, a recirculation system similar to recirculation system 1130 in FIGS. 11 and 12 could be added to the system in FIG. 14.

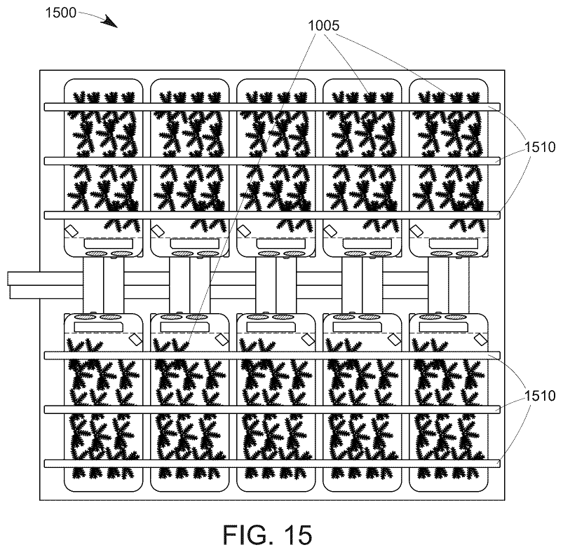

FIG. 15 shows a schematic example of a system 1500 for plant propagation from a top view, including multiple replication capsules 1005, and multiple light sources 1510. The components of the system 1500 in FIG. 15 are similar to those in FIGS. 11-14 described above. The system 1500 in FIG. 15 includes a plurality of replication capsules 1005 (e.g., about ten) coupled together in parallel, with a plurality of light sources 1510 (e.g., about six) positioned above the replication capsules 1005. The light sources 1510 are positioned perpendicular with a major axis of the replication capsules 1005 in this arrangement. A single light source 1510 is positioned over multiple replication capsules 1005 (e.g., five) in this example. The light sources 1510 can be LED light sources, incandescent light sources, or fluorescent light sources, in some examples.

In some cases, the devices, systems, and methods for plant propagation described herein (e.g., those described in FIGS. 7-14) can be used to vegetatively propagate disease-free, young plants similar to or the same as plant tissue culture produced in non-sterile conditions.

In some cases, the replication capsule(s) 1000 or 1005 to house developing vegetative plant material can be made from clear plastic for light transmission with inlets and outlets for liquid flow, an operable opening to add and retrieve plant material, and filtered ventilation to allow for gas egress without exterior contaminate ingress. Replication capsule(s) 1000 or 1005 may be connected in series, parallel, or series-parallel to increase output with shared components, for example as shown in FIGS. 12, 14 and 15.

In some cases, a camera or image sensor (e.g., 1090 in FIG. 10) can be housed within the replication capsule(s) 1000 or 1005, which captures images of the plantlets and the interior of the replication capsule 1000 or 1005. For example, the camera or image sensor can be used to monitor propagation efficiency and plant health, which can be used by the processor to inform automatic protocol adaptations, or which can be used by an operator to make manual adjustments. In some cases, the images from the camera can be used to qualitatively or quantitatively score propagation efficiency and plantlet health. For example, image recognition can be used to translate images to quantitative metrics. In other cases, the images from the camera can be saved and communicated to a user or operator using a communication system.

In some cases, the devices, systems, and methods for plant propagation described herein (e.g., those described in FIGS. 7-14) can use light sources (e.g., LED light fixtures) to deliver a determined lighting spectrum, intensity, and/or photoperiod to aid in the development of vigorous and viable young plants. The processor can be used to change the lighting spectrum, intensity, and photoperiod based on a predetermined protocol, or based on information from one or more sensors. For example, the lighting spectrum, intensity, and photoperiod can be changed during different stages of development (establishment, replication, rooting, and/or acclimatization) of a plantlet.

In some cases, the devices, systems, and methods for plant propagation described herein (e.g., those described in FIGS. 7-14) can include liquid tanks including biostimulant nutrients. The nutrients can include vitamins, minerals, hormones, salts, and other growth-stimulating compounds to facilitate rapid and uniform vegetative plant replication or rooting of vegetatively propagated cuttings. Each liquid tank can contain a pre-mixed solution or individual components which will be combined within the system. The nutrients can be targeted for a particular species of plantlet and/or can be changed during different stages of development (establishment, replication, rooting, and/or acclimatization) of the plantlet.

In some cases, the devices, systems, and methods for plant propagation described herein (e.g., those described in FIGS. 7-14) can include a beneficial microbiota liquid tank to positively influence plant phenotypic expression, enhance growth and vigor, and contribute to a symbiotic microbiome in each capsule such that harmful bacteria and fungi cannot excessively proliferate. Each liquid tank may contain a pre-mixed solution including beneficial microbiota or individual components of beneficial microbiota which will be combined within the system.

In some cases, the devices, systems, and methods for plant propagation described herein (e.g., those described in FIGS. 7-14) can include a gentle cleansing agent solution in a liquid tank to enable periodic or action-induced sanitation of vegetative plant material and the interior of the replication capsule 1000 or 1005. For example, a microbial activity sensor can be used to detect the presence of a microbe or biofilm (or a detrimental microbe or biofilm, if the microbial activity sensor (e.g., sensor 1082 in FIG. 10) is capable of detecting different types of biofilms), and the processor can use information from the microbial activity sensor to deliver the cleansing agent to the replication capsule 1000 or 1005.

In some cases, the devices, systems, and methods for plant propagation described herein (e.g., those described in FIGS. 7-14) can include a reverse osmosis (R.O.) water source to provide clean and consistent rinsing of vegetative plant material and dilution of biostimulant nutrients, beneficial microbiota, and gentle cleansing solutions containing cleansing agents. The systems can use a mixing tank 1210 for mixing and/or dilution, as described above, which may use R.O. water to dilute biostimulant nutrient mixes or beneficial microbiota before flowing into the replication capsule 1000 or 1005.

In some cases, the devices, systems, and methods for plant propagation described herein (e.g., those described in FIGS. 7-14) can include gas tanks containing targeted gases such as carbon dioxide, oxygen, and/or ethylene. The systems can also include a gas mixer 1129, which meters and mixes targeted gases before flowing into the replication capsule 1000 or 1005.

In some cases, the devices, systems, and methods for plant propagation described herein (e.g., those described in FIGS. 7-14) can include a bubble generator 1110, which can form bulk bubbles (e.g., nanobubbles) to stimulate growth in propagating young plants and aid in dispersal of biofilms. The bubble generator 1110 can generate bubbles in liquids including biostimulant nutrients, beneficial microbiota, and/or gentle cleansing solutions. In some cases, different gases may be used to generate bubbles in the different liquids. For example, a mixture of oxygen and ethylene can be added to liquids including nutrients, while oxygen can be used to generate bubbles in a cleansing solution.

In some cases, the devices, systems, and methods for plant propagation described herein (e.g., those described in FIGS. 7-14) can include a fluid sanitation apparatus such as a UV-C light or filter sterilizer. Such systems can be coupled to a fluid collection reservoir (e.g., sanitation system 1240), which houses liquids following immersion cycles before fluid is recirculated into the capsule, before or after sanitation. For example, in systems that use recirculation, the sanitation system 1240 can help remove harmful microorganisms from fluid following immersion cycles and before being recirculated.

In some cases, the devices, systems, and methods for plant propagation described herein (e.g., those described in FIGS. 7-14) can include a processor (e.g., in a computing device, or a server or remote computing device) which controls the system components, controls environmental regulation, collects sensor data, stores and transmits sensor data and captured images with their associated data in a database (e.g., locally or in the cloud). The processor can also control communication systems to transmit alerts to a user or operator, for example by texting, emailing, or via an interface on a computer, tablet, phone or other device.

In some cases, the devices, systems, and methods for plant propagation described herein (e.g., those described in FIGS. 7-14) can include air and/or liquid filters to prevent entry and proliferation of undesired microorganisms within the system. For example, air and liquid filters can be coupled to the gas inlet, gas outlet, liquid inlet and liquid outlet, of the replication capsule 1000 or 1005.

In some cases, the devices, systems, and methods for plant propagation described herein (e.g., those described in FIGS. 7-14) can include fans, air compressors, electronically controlled gas valves, liquid pumps, electronically controlled liquid valves, gas flow meters, and liquid flow meters, to measure and deliver precise levels of fluids and gasses to the replication capsule 1000 or 1005, and regulate gas and liquid exchange within the capsule, and other components of the systems.

In some cases, the devices, systems, and methods for plant propagation described herein (e.g., those described in FIGS. 7-14) can include gaskets, seals, tubes, and various joinery to aid in liquid and gas transfer, prevent leaks, and enable continued function over time.

In some cases, the devices, systems, and methods for plant propagation described herein (e.g., those described in FIGS. 7-14) can include mounting brackets and hardware to consolidate and organize components so that multiple units may be connected in series and/or parallel and easily serviced.

In some cases, the devices, systems, and methods for plant propagation described herein (e.g., those described in FIGS. 7-14) can include one or more sensors, which measure one or more environmental and/or systems process parameters. A processor of the systems can use information from the sensors to control system components and processes, and update system set points and programs, as described herein. For example, sensors can measure environmental parameters and send data to a processor of a control system (e.g., system 600 in FIG. 6), which can compare sensor data with target environment data and activate various components of the system such as fans or heaters to act as environmental regulation mechanisms and re-stabilize the environment to target parameters. In some cases, the control system can alert a user or operator in response to a sensor measurement, and the user or operator can manually change a system set point or program.

In some cases, the devices, systems, and methods for plant propagation described herein (e.g., those described in FIGS. 7-14) can include a temperature sensor positioned to measure a temperature of the interior volume defined by the replication capsule 1000 or 1005. A processor can actuate a gas valve, a fan, and/or air compressor in response to a measurement from the temperature sensor. For example, if a temperature reading is above a predetermined threshold, then the processor can instruct the gas valve to open, and the fan or air compressor to turn on or speed up to deliver more gas to the replication capsule 1000 or 1005. Once the temperature falls below the threshold or enters a predetermined acceptable range then the processor can instruct the gas valve to close, and the fan or air compressor to turn off or slow down to deliver less gas to the replication capsule 1000 or 1005.

In some cases, the devices, systems, and methods for plant propagation described herein (e.g., those described in FIGS. 7-14) can include a humidity sensor positioned to measure a humidity of the interior volume defined by the replication capsule 1000 or 1005, or a vapor pressure deficit sensor positioned to measure a vapor pressure deficit of the interior volume of the replication capsule 1000 or 1005. A processor can actuate a gas valve, a fan, and/or air compressor in response to a measurement from the humidity or vapor pressure deficit sensor. For example, if a humidity reading is above a predetermined threshold, or if the vapor pressure deficit is below a predetermined threshold, then the processor can instruct the gas valve to open, and the fan or air compressor to turn on or speed up to deliver more gas to the replication capsule 1000 or 1005. Once the humidity falls below the threshold or enters a predetermined acceptable range, or once the vapor pressure deficit enters a predetermined acceptable range, then the processor can instruct the gas valve to close, and the fan or air compressor to turn off or slow down to deliver less gas to the replication capsule 1000 or 1005.

In some cases, the devices, systems, and methods for plant propagation described herein (e.g., those described in FIGS. 7-14) can include an electrical conductivity sensor and/or a pH sensor positioned to measure an electrical conductivity and/or a pH of a liquid in a liquid tank, in a liquid mixing tank, or in the replication capsule 1000 or 1005. Electrical conductivity and/or pH can indicate a concentration of a nutrient, microbiota, and/or cleansing agent in a liquid, and a processor can actuate a liquid valve, and/or a liquid pump in response to a measurement from the electrical conductivity sensor. For example, if an electrical conductivity measurement of liquid in a mixing tank is below a predetermined threshold, or if a pH measurement of liquid in a mixing tank is outside of a predetermined range, then the processor can instruct the liquid valve to open, and/or the liquid pump to turn on or speed up to deliver more liquid from a liquid tank to the mixing tank to be dilute with water. Once the electrical conductivity or pH enters a predetermined acceptable range then the processor can instruct the liquid valve to close, and/or the liquid pump to turn off or slow down to deliver less liquid to the mixing tank. In another example, a processor can use electrical conductivity and/or pH measurements of liquid in a replication capsule 1000 or 1005 to determine what liquids from which liquid tanks need to be added to meet an acceptable criterion. In some cases, the processor can take into account the stage of development of the plantlet, and information from the electrical conductivity and/or pH sensor in determining the acceptable range or criteria.

In some cases, the devices, systems, and methods for plant propagation described herein (e.g., those described in FIGS. 7-14) can include a sensor to measure air exchange rate of a gas in the replication capsule 1000 or 1005. For example, an air exchange rate sensor can be coupled to the gas inlet or gas outlet of replication capsule 1000 or 1005. A processor can actuate a gas valve, a fan, and/or air compressor in response to a measurement from the air exchange rate sensor to deliver more or less gas to the replication capsule 1000 or 1005.

In some cases, the devices, systems, and methods for plant propagation described herein (e.g., those described in FIGS. 7-14) can include a gas composition sensor positioned to measure a carbon dioxide, oxygen, or ethylene gas concentration in the interior volume of the replication capsule 1000 or 1005, or in a gas mixer. A processor can actuate a gas valve, a fan, and/or air compressor in response to a measurement from the gas composition sensor. For example, if a carbon dioxide reading in an gas mixer or in the replication capsule 1000 or 1005 is below a predetermined threshold, then the processor can instruct the gas valve between the carbon dioxide tank and the gas mixer or the replication capsule 1000 or 1005 to open, and optionally a fan or air compressor to turn on or speed up, to deliver more carbon dioxide gas to the gas mixer or replication capsule 1000 or 1005. Once the carbon dioxide concentration enters a predetermined acceptable range, then the processor can instruct the gas valve to close, and optionally the fan or air compressor to turn off or slow down, to deliver less carbon dioxide gas to the gas mixer or replication capsule 1000 or 1005. In another example, information from a gas composition sensor can be used by the processor to open or close valves and adjust the composition of gas that is used by a bubble generator (e.g., nanobubble generator) of the system.

In some cases, the devices, systems, and methods for plant propagation described herein (e.g., those described in FIGS. 7-14) can include a gas composition sensor positioned to measure a carbon dioxide, oxygen, or ethylene gas concentration entering the replication capsule 1000 or 1005 and a gas composition sensor positioned to measure a carbon dioxide, oxygen, or ethylene gas concentration exiting the replication capsule 1000 or 1005. The processor can use information about the difference in gases entering and exiting the replication capsule 1000 or 1005 to infer about gases being consumed or generated within the replication capsule 1000 or 1005. For example, the difference between carbon dioxide concentration entering the replication capsule 1000 or 1005 and exiting the replication capsule 1000 or 1005 can be used as an indicator of plantlet growth. The processor can then use such information to determine how to control the system. For example, the processor can determine that a plantlet has entered a new stage of development, and instruct the system to change parameters, set points, acceptable ranges, and/or protocols accordingly.

In some cases, the devices, systems, and methods for plant propagation described herein (e.g., those described in FIGS. 7-14) can include a light sensor positioned to measure a light intensity, light spectrum, and/or light photoperiod of the light illuminating the replication capsule 1000 or 1005. For example, the light sensor can be positioned inside replication capsule 1000 or 1005, or on the outside of replication capsule 1000 or 1005 facing the illumination source. A processor can control the light source based on information from the light sensor. For example, if a measured light intensity is below a predetermined threshold, then the processor can instruct the light source to increase the output for example by providing more electrical current to the light source. Once the light intensity is within an acceptable range, then the processor can instruct the light source to maintain the output. In some cases, the processor can take into account the stage of development of the plantlet, and information from the light sensor in determining the acceptable range.

In some cases, the devices, systems, and methods for plant propagation described herein (e.g., those described in FIGS. 7-14) can include one or more liquid level sensors to measure a volume of liquid in a liquid tank, in a mixing tank, and/or in the replication capsule 1000 or 1005. A processor can actuate a liquid valve, and/or a liquid pump in response to a measurement from the liquid level sensor to deliver liquid until an acceptable volume of liquid is detected in the liquid tank, in a mixing tank, and/or in the replication capsule 1000 or 1005.

In some cases, the devices, systems, and methods for plant propagation described herein (e.g., those described in FIGS. 7-14) can include microbial activity sensor positioned to measure a microbial activity in the replication capsule 1000 or 1005. Microbial activity sensors can indicate the presence of detrimental microbiota, and a processor can initiate a protocol based on information from the microbial activity sensors. For example, a processor can initiate a disinfection protocol wherein one or more liquid valves, and/or a liquid pump are controlled to deliver a cleansing agent or a liquid containing beneficial microbiota to the replication capsule 1000 or 1005. In some cases, the disinfection protocol can include multiple blocks, such as adding a cleansing agent to replication capsule 1000 or 1005, draining the cleansing agent from replication capsule 1000 or 1005, rising replication capsule 1000 or 1005 with R.O. water, and then adding beneficial microbiota to replication capsule 1000 or 1005. Once the microbial activity sensor indicates that the microbial activity is in an acceptable range or criteria, then the processor can instruct the system to resume a growth protocol including delivering liquid with nutrients to the replication capsule 1000 or 1005. In some cases, the processor can receive a stage of development of the plantlet, and information from the microbial activity sensor to determine an acceptable range or criteria. For example, in the establishment stage, the plantlet may be more susceptible to infection, and the acceptable range or criteria is more stringent than in later stages of plantlet development.

In some embodiments, a protocol can be used by a processor to regulate the relative humidity (RH) in a replication capsule of the devices, systems, and methods described herein. For example, a replication capsule like replication capsule 1000 in FIG. 10, and a control system like system 600 in FIG. 6 can be used. For example, the protocol can specify an acceptable range of the RH of the replication capsule (e.g., from about 80% RH to about 85% RH).

The set point in the protocol can be specified to meet a minimum RH (e.g., about 80% RH) and a maximum RH (e.g., about 85% RH). Sensor readings can be taken once every 5 minutes using a humidity sensor that measures humidity within the replication capsule. The following timeline describes an example of applying a control protocol using the processor.

Minute 0: Fan is off. The humidity sensor within replication capsule (e.g., 610 in FIG. 6) is read, the humidity sensor data is recorded (e.g., to database 660 in FIG. 6), and the data is relayed to the interface/set point (e.g., input interface 640 in FIG. 6) using the processor. The processor then checks the humidity data against the set point and the acceptable range. The processor determined that the RH is within an acceptable range and sends a signal to the control board (e.g., 620 in FIG. 6) to maintain current state (fan off).

Minute 5: The humidity sensor within the replication capsule is read a second time, the humidity sensor data is recorded, relayed to the interface/set point, and checked against the set point using the processor. At this time, the processor can determine that the RH is above the acceptable range and sends a signal to the control board to change the current state of the fan to on. The control board can then activate the fan to turn it on.

Minute 10: Fan is still on. The humidity sensor within replication capsule is read a third time, the humidity sensor data is recorded, relayed to the interface/set point, and checked against the set point and the acceptable range using the processor. The processor determines at this time that the RH is below the acceptable range and sends a signal to the control board to change the current state of the fan to off. The control board can then deactivate the fan to turn it off.

In some embodiments, a protocol can be used by a processor to regulate the delivery of a liquid containing a nutrient to a replication capsule of the devices, systems, and methods described herein. For example, a system like those shown in FIGS. 8 and 11 can be used. The protocol can specify that a volume (e.g., 0.5 L) of a liquid containing a nutrient (i.e., a nutrient solution) flood into the replication capsule according to a schedule (e.g., every 4 hours), remain in the capsule for a duration (e.g., 30 minutes), then drain from the replication capsule. The following timeline describes an example of applying a control protocol using the processor.

Minute 50:10 minutes before nutrient flood is scheduled to occur. Electronic valves from an R.O. water tank and nutrient tank(s) pull metered doses of liquids to equal 0.5 L into the mixing and dilution tank and stir to incorporate.

Minute 60 (Hour 1): Nutrient flood is scheduled to occur. The electronic valve from the mixing tank releases a liquid containing nutrients, which is a diluted nutrient solution in this example, into the replication capsule. Upon a liquid level sensor in the replication capsule sensing liquid immersion, the data from the liquid level sensor is recorded to a database, and the processor sends a signal to the control board to a start timer for an electronic liquid release valve coupled to the drain of the replication capsule to remain closed for 30 minutes.

Minute 90: After the 30 minutes elapses, the electronic liquid release valve is opened, and the liquid drains from the replication capsule. The liquid level sensor then senses a lack of liquid immersion, and the data is recorded to the database.

Hour 3+50 minutes: According to a schedule of the protocol, the nutrient dilution and mixing process performed at minute 50 can be repeated.

Hour 4: The nutrient flood process performed at hour 1 is repeated.

Hour 4+30 minutes: The liquid drain process performed at minute 90 is repeated.

In some embodiments, the devices, systems, and methods described herein can be used to experimentally test two protocols (Protocol A and Protocol B) and compare the results to determine which protocol yields better results for plant propagation. The system used for this example used a system like those shown in FIGS. 7-14. The starting plant material can be vegetative cuttings of a plant. Each protocol can include supplying a liquid from a liquid tank containing a biochemical and nutrient formula, and maintaining environmental conditions, such as temperature, humidity, gas composition, and lighting conditions.

The liquid delivered to the replication capsules can include one or more of the following biochemical and nutrient species mixed with R.O water., Ferric Sodium EDDHA, Cupric Sulfate, Sodium Molybdate, Manganese Chloride, Zinc Sulfate, Boric Acid, Magnesium Sulfate, Potassium Nitrate, Potassium Iodide, Ammonium Phosphate, Calcium Nitrate, Calcium Chloride, Thiamine Hydrochloride, Pyridoxine Hydrochloride, Nicotinic Acid, L-Glutamine, L-Ascorbic Acid, Casein Hydrolysate, Sucrose, and other biochemicals to stimulate growth.

The environmental parameters of the replication capsule can be controlled using a processor according to acceptable ranges or set points. Some examples of environmental parameters of the replication capsule include, day temperature (e.g., from about 60° F. to about 80° F.), night temperature (e.g., from about 50° F. to about 70° F.), humidity (e.g., from about 70% to about 95%, vapor pressure deficit (e.g., from about 0.5 kPa to about 1.5 kPa), electrical conductivity of the liquid in the replication capsule (e.g., from about 1000 μS/cm to about 5000 μS/cm), pH of the liquid in the replication capsule (e.g., from about 5 pH to about 7 pH), air exchange rate (e.g., from about 0.5 to about 2 times per hour), ethylene concentration (e.g., from about 0.01 to about 0.1 ppm), CO₂ concentration (e.g., from about 100 ppm to about 1000 ppm), O₂ concentration (e.g., from about 10 ppm to about 1000 ppm), light intensity (e.g., from about 1000 to about 10000 lux), and photoperiod (e.g., from about 8 to about 18 hours).

The processor can control the system to deliver a liquid mixture to the replication capsule according to a nutrient flood interval and volume schedule.

The processor can also control the system to deliver a liquid containing microbiota (e.g., probiotic bacteria and/or communities of mycorrhizal soil microorganisms) according to an injection interval and volume schedule.

In some embodiments, the processor can control the nutrient liquids and environmental parameters according to protocols (e.g., in parallel replication capsules) in order to compare different protocols. The results of the protocols can be qualitatively assessed in different categories using a scoring system, and the scores can be combined into an aggregate score to compare one protocol with another. For example, plantlet health can be evaluated using the categories of plantlet morphology, the presence of hyperhydricity, coloration, propagation rate, bacterial contamination level, fungal contamination level, survival rate, and root development. The following is an example of a qualitative scoring key in different categories, where in all cases, a higher qualitative score indicates a better result.

Morphology:
1—Poor: High level of abnormal structures
2—Moderate: Some abnormal structures only present in old growth
3—Good: No abnormal structures

Presence of hyperhydricity:
1—Poor: All tissue nearly translucent and fragile
2—Moderate: Some translucent tissue, mainly observed in older growth
3—Good: No translucent tissue

Coloration:
1—Poor: Large amounts of tissue displaying black tissue necrosis, yellow, red, or brown coloration
2—Moderate: Small amounts of tissue showing discoloration with new growth being healthy
3—Good: Even coloration throughout

Propagation rate:
≤1× per month: Poor, lack of new shoots or roots, no expansion or shriveled appearance
2× per month—Low
3× per month—Moderate
4× per month—Good
≥5× per month—Excellent
≥10× per month—Exceptional

Detrimental microbial level:
1—Poor: Presence of fast-growing fungal mycelium or bacterial colonies causing biofilm buildup and plant death
2—Moderate: Some fast growing fungal mycelium or bacteria present but maintained by replicator controls and no loss of tissue viability
3—Good: No detrimental microbial (fungal or bacterial) presence detected

Survival rate:
1—Poor: 50% or less of plant material is viable
2—Moderate: 75% or more of plant material is viable
3—Good: 90% or more of plant material is viable

Root development:
1—Poor: No root development or little development with abnormal formation or coloration
2—Moderate: Some development of healthy roots
3—Good: High number of robust, well formed, evenly colored roots with extensive branching

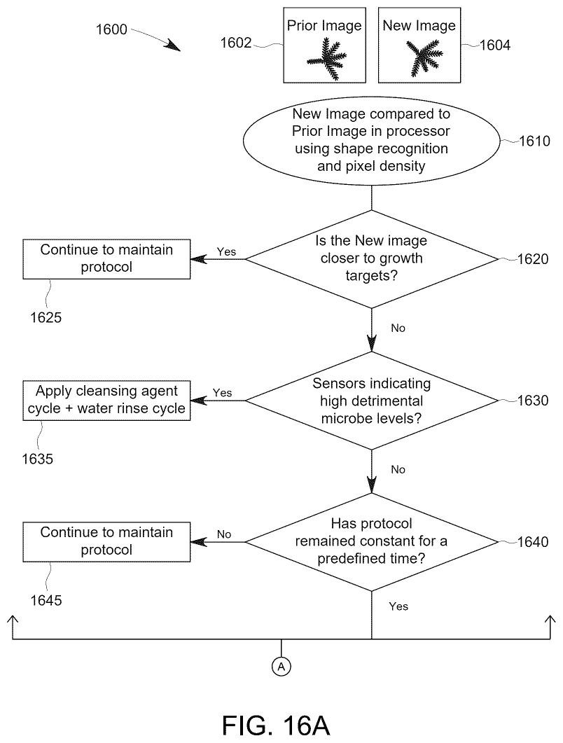

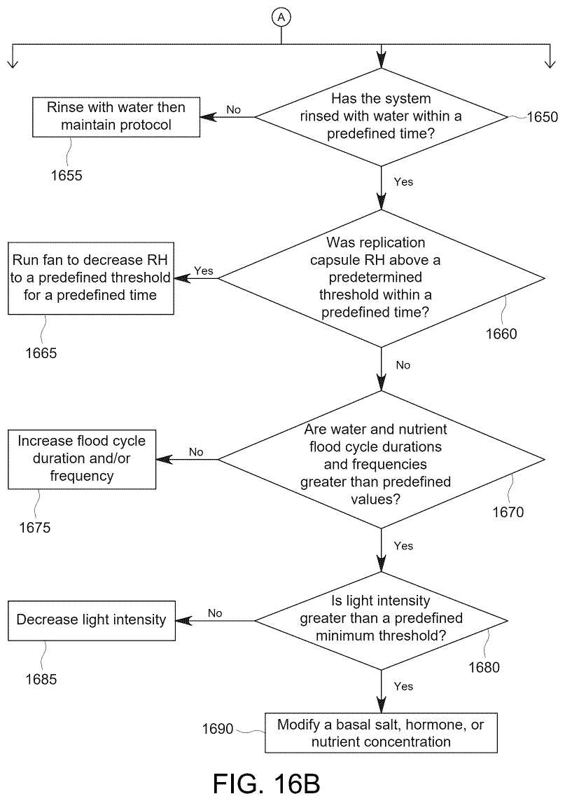

FIGS. 16A and 16B show an example of a decision tree 1600 with feedback loops using the devices, systems, and methods described herein and plantlet photography, in accordance with some embodiments. In this embodiment, a prior image 1602 may be saved in memory coupled to the processor. New image 1604 may be taken using a camera of a replication capsule described herein (e.g., image sensor 1090 in FIG. 10).

In block 1610, the new image 1604 may be compared with the prior image 1602 saved in memory. In some embodiments, the processor can analyze the new image 1604 and the prior image 1602 and determine differences between the images in different categories using one or more image recognition algorithms.

In block 1620, the new image 1604 and the prior image 1602 may be compared using the processor to determine whether the new image 1604 may be closer to predetermined growth targets (e.g., in the categories described above). If the new image 1604 is determined to be closer to predetermined growth targets, then the current protocol is maintained in block 1625. If not, then decision tree 1600 proceeds to block 1630.

In block 1630, a sensor of the system (e.g., sensor 1082 of FIG. 10) may output information to the processor about detrimental microbe levels in the replication capsule. If the detrimental microbe levels are determined to be too high based on the information from the sensor, then the processor instructs the system to apply a cleansing agent and a water rinse cycle. If not, then decision tree 1600 proceeds to block 1640.

In block 1640, the processor of the system may determine whether the protocol has remained constant for a predefined period of time (e.g., 1 or more days, 4 or more days, or more than 7 days). When the protocol has not remained constant for the predefined period of time, then the processor may instruct, or output a signal to, the system to maintain the current protocol in block 1645. If the protocol has remained constant for the predefined period of time, then the process proceeds to block 1650.

In block 1650, the processor determines whether the replication capsule has been rinsed (e.g., with water) within a predefined period of time (e.g. in the last 6 hours, in the last 12 hours, in the last 24 hours, or in the last 3 days). If the system has not been rinsed (e.g., with water) within the predefined period of time then the processor instructs the system to rinse the replication capsule with water and then to maintain the current protocol in block 1655. If the protocol has been rinsed (e.g., with water) within the predefined period of time, then the decision tree 1600 proceeds to block 1660.

In block 1660, a sensor of the system (e.g., sensor 1080 or 1082 of FIG. 10) provides information to the processor about relative humidity levels in the replication capsule. If the relative humidity (RH) in the replication capsule was higher than a predefined threshold (e.g., 70% RH, 80% RH, or 90% RH) within a predefined time (e.g., in the last 24 hours), then the processor can instruct the system to run a fan as needed to decrease the relative humidity (e.g., to 60% RH, 70% RH, or 80% RH) in block 1665. If the relative humidity in the replication capsule was not higher than the predefined threshold within the predefined time, then decision tree 1600 proceeds to block 1670.

In block 1670, the processor determines if the water and nutrient flood cycles have durations greater than a predefined time (e.g., 10 minutes, 30 minutes, or 1 hour), and if the water and nutrient flood cycles have frequencies greater than a predefined value (e.g., 2 times per day, 4 times per day, or more than 4 times per day). In some cases, one or more sensors (e.g., sensors 1080 or 1082 in FIG. 10) can be used to determine the flood durations. If the water and nutrient flood cycles do not have durations greater than the predefined time duration and/or frequencies greater than the predefined value, then the processor instructs the system to increase the flood cycle duration and/or frequency in block 1675. If the water and nutrient flood cycles do have durations greater than the predefined time duration and frequencies greater than the predefined value, then the decision tree 1600 proceeds to block 1680.

In block 1680, a sensor of the system (e.g., sensor 1080 or 1082 of FIG. 10) provides information to the processor about light intensity in the replication capsule. If the light intensity is not greater than a predefined minimum threshold (e.g., 2000 LUX, 2500 LUX, or 3000 LUX), then the processor can instruct a light source of the system to increase the light intensity in block 1665. If the light intensity is greater than the predefined minimum threshold, then decision tree 1600 proceeds to block 1690.

In block 1690 a basal salt, hormone, or nutrient concentration is changed, for example, by a predefined amount (e.g., up to 10%, up to 20%, up to 50%, or up to 100%, or more than 100%).

### EMBODIMENTS

Clause 1. A plant tissue culture apparatus comprising: a replication capsule comprising a capsule body, liquid inlets, gas inlets, a gas outlet, and a liquid outlet, wherein an internal volume defined by the capsule body is configured to contain a plurality of plantlets; a light source configured to illuminate the plurality of plantlets in the replication capsule; a first gas tank coupled to the gas inlet and configured to contain a first gas; a first liquid tank coupled to the liquid inlet, the first liquid tank configured to contain a first liquid mixture; a nanobubble generator positioned between the first liquid tank and the liquid inlet; a second gas tank configured to contain a second gas, wherein the second gas tank is coupled to the nanobubble generator, wherein the nanobubble generator is configured to generate bubbles comprising the second gas in the first liquid mixture; a liquid valve between the first liquid tank and the liquid inlet; a first gas valve between the first gas tank and the gas inlet; a second gas valve between the second gas tank and the nanobubble generator; and a processor coupled to the liquid valve, the first gas valve, the second gas valve, and the light source, wherein the processor is configured to control the liquid valve, the first gas valve, the second gas valve, and the light source based on predetermined parameters or in response to information from a sensor.

Clause 2. The plant tissue culture apparatus of clause 1, wherein the first liquid mixture comprises nutrients, and wherein the bubbles generated by the nanobubble generator comprise air with excess oxygen.

Clause 3. The plant tissue culture apparatus of clause 1, wherein the first gas comprises carbon dioxide, and wherein the second gas comprises oxygen.

Clause 4. The plant tissue culture apparatus of clause 1, wherein the bubbles generated by the nanobubble generator comprise nanobubbles.

Clause 5. The plant tissue culture apparatus of clause 1, further comprising a fan or air compressor coupled to a gas mixer, wherein the first gas tank is coupled to the gas mixer, and wherein the gas mixer is coupled to the gas inlet.

Clause 6. The plant tissue culture apparatus of clause 1, further comprising a liquid pump positioned between the first liquid tank and the liquid inlet.

Clause 7. The plant tissue culture apparatus of clause 1, wherein the first liquid tank and the liquid inlet are configured such that the first liquid mixture can be introduced into the replication capsule through the liquid inlet using a force of gravity.

Clause 8. The plant tissue culture apparatus of clause 1, further comprising a water source, configured to provide filtered water, and a mixing tank, wherein: the water source is coupled to the mixing tank, the first liquid tank is coupled to the mixing tank, the mixing tank is configured to dilute the first liquid mixture with the filtered water from the water source, and the nanobubble generator is positioned between the mixing tank and the liquid inlet.

Clause 9. The plant tissue culture apparatus of clause 1, further comprising a second liquid tank coupled to the liquid inlet, wherein: the second liquid tank is configured to contain a second liquid mixture, the nanobubble generator is further positioned between the second liquid tank and the liquid inlet, and the nanobubble generator is additionally configured to generate bubbles of the second gas in the second liquid mixture.

Clause 10. The plant tissue culture apparatus of clause 9, wherein the first liquid mixture comprises nutrients, wherein the second liquid mixture comprises microbiota, wherein the first gas comprises air with excess carbon dioxide, and wherein the second gas comprises air with excess oxygen.

Clause 11. The plant tissue culture apparatus of clause 10, further comprising a third liquid tank coupled to the liquid inlet, wherein the third liquid tank is configured to contain a third liquid mixture, wherein: the nanobubble generator is further positioned between the third liquid tank and the liquid inlet, the nanobubble generator is configured to generate bubbles of the second gas in the third liquid mixture, and the third liquid mixture comprises a cleansing agent.

Clause 12. The plant tissue culture apparatus of clause 1, further comprising: a mixing tank coupled to the liquid inlet; a water source that is configured to provide filtered water coupled to the mixing tank; and a plurality of liquid tanks coupled to the mixing tank, wherein the first liquid tank is one of a plurality of liquid tanks, wherein the plurality of liquid tanks each comprise a nutrient or a solution comprising microbiota; wherein the nanobubble generator is further positioned between the mixing tank and the liquid inlet, and wherein the nanobubble generator is configured to generate bubbles of the second gas in liquid from the mixing tank.

Clause 13. The plant tissue culture apparatus of clause 1, further comprising a second replication capsule comprising a second capsule body, a second liquid inlet, a second gas inlet, a second gas outlet, and a second liquid outlet, wherein an internal volume defined by the second capsule body is configured to contain a plurality of plantlets, wherein the first gas tank is coupled to the second gas inlet, and wherein the first liquid tank is coupled to the second liquid inlet.

Clause 14. The plant tissue culture apparatus of clause 1, wherein the processor is further configured to control one or more of an intensity of the light source, a spectrum of the light source, or a photoperiod of the light source.

Clause 15. The plant tissue culture apparatus of clause 1, wherein the replication capsule further comprises a membrane positioned between the gas inlet and the plurality of plantlets, and between the liquid inlet and the plurality of plantlets.

Clause 16. The plant tissue culture apparatus of clause 1, wherein the replication capsule further comprises a detachable cap, wherein the detachable cap comprises the gas inlet and the liquid inlet.

Clause 17. The plant tissue culture apparatus of clause 1, further comprising the sensor, wherein the processor is configured to control the liquid valve, the first gas valve, the second gas valve, and the light source based on information from the sensor, and wherein the sensor is one of: a temperature sensor configured to detect a temperature in the replication capsule, a humidity sensor configured to measure a humidity of the replication capsule, a vapor pressure deficit sensor configured to measure a vapor pressure deficit of the replication capsule, an electrical conductivity sensor configured to measure an electrical conductivity of the first liquid mixture, a pH sensor configured to measure a pH of the first liquid mixture, an air exchange rate sensor configured to measure an air exchange rate of the replication capsule, a carbon dioxide sensor configured to measure a carbon dioxide level of the replication capsule, an oxygen sensor configured to measure an oxygen level of the replication capsule, an ethylene sensor configured to measure an ethylene level of the replication capsule, a light sensor configured to measure light intensity level of illumination in the replication capsule, a liquid level sensor configured to measure a liquid level of the first liquid mixture in the first liquid tank or in the replication capsule, a gas flow meter configured to measure a flow of a gas between the first gas tank and the gas inlet, a liquid flow meter configured to measure a flow of a liquid between the first liquid tank and the liquid inlet, and a microbial activity sensor configured to measure microbial activity in the replication capsule.

Clause 18. The plant tissue culture apparatus of clause 1, wherein the sensor is a temperature sensor configured to detect a temperature in the replication capsule, and wherein the processor is configured to control the liquid valve, the first gas valve, the second gas valve, or the light source based on information from the temperature sensor.

Clause 19. The plant tissue culture apparatus of clause 1, wherein the sensor is a humidity sensor configured to measure a humidity of the replication capsule, and wherein the processor is configured to control the liquid valve, the first gas valve, the second gas valve, or the light source based on information from the humidity sensor.

Clause 20. The plant tissue culture apparatus of clause 1, wherein the sensor is a vapor pressure deficit sensor configured to measure a vapor pressure deficit of the replication capsule, and wherein the processor is configured to control the liquid valve, the first gas valve, the second gas valve, or the light source based on information from the vapor pressure deficit sensor.

Clause 21. The plant tissue culture apparatus of clause 1, wherein the sensor is an electrical conductivity sensor configured to measure an electrical conductivity of the first liquid mixture, and wherein the processor is configured to control the liquid valve, the first gas valve, the second gas valve, or the light source based on information from the electrical conductivity sensor.

Clause 22. The plant tissue culture apparatus of clause 1, wherein the sensor is a pH sensor configured to measure a pH of the first liquid mixture, and wherein the processor is configured to control the liquid valve, the first gas valve, the second gas valve, or the light source based on information from the pH sensor.

Clause 23. The plant tissue culture apparatus of clause 1, wherein the sensor is an air exchange rate sensor configured to measure an air exchange rate of the replication capsule, wherein the processor is configured to control the liquid valve, the first gas valve, the second gas valve, or the light source based on information from the air exchange rate sensor.

Clause 24. The plant tissue culture apparatus of clause 1, wherein the sensor is a carbon dioxide sensor configured to measure a carbon dioxide level of the replication capsule, and wherein the processor is configured to control the liquid valve, the first gas valve, the second gas valve, or the light source based on information from the carbon dioxide sensor.

Clause 25. The plant tissue culture apparatus of clause 1, wherein the sensor is an oxygen sensor configured to measure an oxygen level of the replication capsule, and wherein the processor is configured to control the liquid valve, the first gas valve, the second gas valve, or the light source based on information from the oxygen sensor.

Clause 26. The plant tissue culture apparatus of clause 1, wherein the sensor is an ethylene sensor configured to measure an ethylene level of the replication capsule, wherein the processor is configured to control the liquid valve, the first gas valve, the second gas valve, or the light source based on information from the ethylene sensor.

Clause 27. The plant tissue culture apparatus of clause 1, wherein the sensor is a light sensor configured to measure light intensity level of illumination in the replication capsule, wherein the processor is configured to control the liquid valve, the first gas valve, the second gas valve, or the light source based on information from the light sensor.

Clause 28. The plant tissue culture apparatus of clause 1, wherein the sensor is a liquid level sensor configured to measure a liquid level of the first liquid mixture in the first liquid tank or in the replication capsule, and wherein the processor is configured to control the liquid valve, the first gas valve, the second gas valve, or the light source based on information from the liquid level sensor.

Clause 29. The plant tissue culture apparatus of clause 1, wherein the sensor is a gas flow meter configured to measure a flow of a gas between the first gas tank and the gas inlet, wherein the processor is configured to control the liquid valve, the first gas valve, the second gas valve, or the light source based on information from the gas flow meter.

Clause 30. The plant tissue culture apparatus of clause 1, wherein the sensor is a liquid flow meter configured to measure a flow of a liquid between the first liquid tank and the liquid inlet, wherein the processor is configured to control the liquid valve, the first gas valve, the second gas valve, or the light source based on information from the liquid flow meter.

Clause 31. The plant tissue culture apparatus of clause 1, wherein the sensor is a microbial activity sensor configured to measure microbial activity in the replication capsule, and wherein the processor is configured to control the liquid valve, the first gas valve, the second gas valve, or the light source based on information from the microbial activity sensor.

Clause 32. The plant tissue culture apparatus of clause 1, further comprising a waste tank coupled to the liquid outlet, wherein the waste tank is not coupled to the liquid inlet using a recirculation system.

Clause 33. The plant tissue culture apparatus of clause 1, further comprising a sanitation tank coupled to the liquid outlet and a recirculation line coupling the sanitation tank to the liquid inlet, wherein the sanitation tank comprises a UV light, chemical treatment, or heat to sterilize the liquid in the sanitation tank.

Clause 34. The plant tissue culture apparatus of clause 1, further comprising a plurality of replication capsules, wherein the plurality of replication capsules is coupled to the first gas tank and to the first liquid tank in a parallel configuration, in a series configuration, or in a combination parallel and series configuration.

Clause 35. The plant tissue culture apparatus of clause 1, wherein the replication capsule further comprises an antimicrobial coating.

Clause 36. The plant tissue culture apparatus of clause 1, further comprising a third gas tank, wherein the third gas tank comprises a third gas.

Clause 37. The plant tissue culture apparatus of clause 1, further comprising a gas mixer, wherein the first gas tank and the second gas tank are coupled to the gas mixer, and wherein the gas mixer is coupled to the gas inlet and to the nanobubble generator.

Clause 38. The plant tissue culture apparatus of clause 37, further comprising a third valve and a fourth valve, wherein the first valve is between the first gas tank and the gas mixer, wherein the second valves is between the second gas tank and the gas mixer, wherein the third valve is between the gas mixer and the replication capsule, and wherein the fourth valve is between the gas mixer and the nanobubble generator, wherein the processor is coupled to the third and fourth valves, and wherein the processor is configured to control the third and fourth valves based on predetermined parameters or in response to information from a sensor.

Clause 39. A method of propagation of a plantlet comprising: introducing a first gas from a first gas tank to a gas inlet of a replication capsule using a first gas valve, the replication capsule comprising a plurality of plantlets; generating bubbles of a second gas in a first liquid mixture using a nanobubble generator and a second gas tank coupled to the nanobubble generator using a second gas valve; introducing the first liquid mixture from the nanobubble generator to a liquid inlet of a replication capsule using a first liquid valve; after a first period of time, draining the first liquid mixture from the replication capsule using a second liquid valve coupled to a liquid outlet of the replication capsule; illuminating the plurality of plantlets with a light source; and controlling the first liquid valve, the first gas valve, the second gas valve, and the light source using a processor coupled to the first liquid valve, the first gas valve, the second gas valve, and the light source, based on predetermined parameters or in response to information from a sensor.

Clause 40. The method of propagation of a plantlet of clause 39, further comprising physically agitating a biofilm in the replication capsule using the bubbles in the first liquid mixture.

Clause 41. The method of propagation of a plantlet of clause 39, further comprising: after a second period of time, repeating a flooding and draining cycle including the introducing the first liquid mixture to the replication capsule and the draining the first liquid mixture from the replication capsule using the first and second liquid valves; and repeating the flooding and draining cycle for a predetermined number of times or until a signal from a sensor is received by the processor.

Clause 42. The method of propagation of a plantlet of clause 39, further comprising: generating bubbles of a second gas in a second liquid mixture using the nanobubble generator and the second gas tank coupled to the nanobubble generator using a second gas valve; and introducing the second liquid mixture from the nanobubble generator to the liquid inlet of the replication capsule using a second liquid valve; wherein the first liquid mixture comprises nutrients, wherein the second liquid mixture comprises microbiota, wherein the first gas comprises carbon dioxide, and wherein the second gas comprises oxygen.

Clause 43. The method of propagation of a plantlet of clause 39, further comprising changing the controlling of one or more of: the first liquid valve, the first gas valve, the second gas valve, or the light source using the processor over time based on information from the sensor, wherein the information from the sensor is used to determine a stage of development of a plantlet of the plurality of plantlets.

Clause 44. The method of propagation of a plantlet of clause 43, wherein the sensor comprises a carbon dioxide sensor coupled to the gas inlet and a carbon dioxide sensor coupled to a gas outlet of the replication capsule, and wherein the information from the sensor that is used to determine the stage of development of the plantlet of the plurality of plantlets is a difference between carbon dioxide concentration of gas entering the replication capsule and gas leaving the replication capsule.

Clause 45. The method of propagation of a plantlet of clause 39, wherein the first liquid mixture comprises nutrients, and wherein the bubbles generated by the nanobubble generator comprise air with excess oxygen.

Clause 46. The method of propagation of a plantlet of clause 39, wherein the first gas comprises carbon dioxide, and wherein the second gas comprises oxygen.

Clause 47. The method of propagation of a plantlet of clause 39, wherein the bubbles generated by the nanobubble generator comprise nanobubbles.

Clause 48. The method of propagation of a plantlet of clause 39, further comprising controlling a fan or air compressor using a processor, wherein the fan or air compressor is coupled to a gas mixer, wherein the first gas tank is coupled to the gas mixer, and wherein the gas mixer is coupled to the gas inlet.

Clause 49. The method of propagation of a plantlet of clause 39, further comprising controlling a liquid pump using the processor, wherein the liquid pump is positioned between the first liquid tank and the liquid inlet.

Clause 50. The method of propagation of a plantlet of clause 39, wherein the first liquid tank and the liquid inlet are configured such that the first liquid mixture can be introduced into the replication capsule through the liquid inlet using the force of gravity.

Clause 51. The method of propagation of a plantlet of clause 39, further comprising: providing filtered water to a mixing tank; providing the first liquid mixture to the mixing tank; diluting the first liquid mixture with water in the mixing tank; and generating bubbles in the water and the first liquid mixture from the mixing tank using the nanobubble generator.

Clause 52. The method of propagation of a plantlet of clause 39, further comprising controlling the first liquid valve, the first gas valve, the second gas valve, and the light source using a processor coupled to the first liquid valve, the first gas valve, the second gas valve, and the light source, based on predetermined parameters or in response to information from a sensor, wherein the sensor is one of: a temperature sensor configured to detect a temperature in the replication capsule, a humidity sensor configured to measure a humidity of the replication capsule, a vapor pressure deficit sensor configured to measure a vapor pressure deficit of the replication capsule, an electrical conductivity sensor configured to measure an electrical conductivity of the first liquid mixture, a pH sensor configured to measure a pH of the first liquid mixture, an air exchange rate sensor configured to measure an air exchange rate of the replication capsule, a carbon dioxide sensor configured to measure a carbon dioxide level of the replication capsule, an oxygen sensor configured to measure an oxygen level of the replication capsule, an ethylene sensor configured to measure an ethylene level of the replication capsule, a light sensor configured to measure light intensity level of illumination in the replication capsule, a liquid level sensor configured to measure a liquid level of the first liquid mixture in the first liquid tank or in the replication capsule, a gas flow meter configured to measure a flow of a gas between the first gas tank and the gas inlet, a liquid flow meter configured to measure a flow of a liquid between the first liquid tank and the liquid inlet, and a microbial activity sensor configured to measure microbial activity in the replication capsule.

Clause 53. The method of propagation of a plantlet of clause 39, wherein the liquid valve, the first gas valve, the second gas valve, or the light source are controlled based on information from the sensor, wherein the sensor is a temperature sensor configured to detect a temperature in the replication capsule.

Clause 54. The method of propagation of a plantlet of clause 39, wherein the liquid valve, the first gas valve, the second gas valve, or the light source are controlled based on information from the sensor, wherein the sensor is a humidity sensor configured to measure a humidity of the replication capsule.

Clause 55. The method of propagation of a plantlet of clause 39, wherein the liquid valve, the first gas valve, the second gas valve, or the light source are controlled based on information from the sensor, wherein the sensor is a vapor pressure deficit sensor configured to measure a vapor pressure deficit of the replication capsule.

Clause 56. The method of propagation of a plantlet of clause 39, wherein the liquid valve, the first gas valve, the second gas valve, or the light source are controlled based on information from the sensor, wherein the sensor is an electrical conductivity sensor configured to measure an electrical conductivity of the first liquid mixture.

Clause 57. The method of propagation of a plantlet of clause 39, wherein the liquid valve, the first gas valve, the second gas valve, or the light source are controlled based on information from the sensor, wherein the sensor is a pH sensor configured to measure a pH of the first liquid mixture.

Clause 58. The method of propagation of a plantlet of clause 39, wherein the liquid valve, the first gas valve, the second gas valve, or the light source are controlled based on information from the sensor, wherein the sensor is an air exchange rate sensor configured to measure an air exchange rate of the replication capsule.

Clause 59. The method of propagation of a plantlet of clause 39, wherein the liquid valve, the first gas valve, the second gas valve, or the light source are controlled based on information from the sensor, wherein the sensor is a carbon dioxide sensor configured to measure a carbon dioxide level of the replication capsule.

Clause 60. The method of propagation of a plantlet of clause 39, wherein the liquid valve, the first gas valve, the second gas valve, or the light source are controlled based on information from the sensor, wherein the sensor is an oxygen sensor configured to measure an oxygen level of the replication capsule.

Clause 61. The method of propagation of a plantlet of clause 39, wherein the liquid valve, the first gas valve, the second gas valve, or the light source are controlled based on information from the sensor, wherein the sensor is an ethylene sensor configured to measure an ethylene level of the replication capsule.

Clause 62. The method of propagation of a plantlet of clause 39, wherein the liquid valve, the first gas valve, the second gas valve, or the light source are controlled based on information from the sensor, wherein the sensor is a light sensor configured to measure light intensity level of illumination in the replication capsule.

Clause 63. The method of propagation of a plantlet of clause 39, wherein the liquid valve, the first gas valve, the second gas valve, or the light source are controlled based on information from the sensor, wherein the sensor is a liquid level sensor configured to measure a liquid level of the first liquid mixture in the first liquid tank or in the replication capsule.

Clause 64. The method of propagation of a plantlet of clause 39, wherein the liquid valve, the first gas valve, the second gas valve, or the light source are controlled based on information from the sensor, wherein the sensor is a gas flow meter configured to measure a flow of a gas between the first gas tank and the gas inlet.

Clause 65. The method of propagation of a plantlet of clause 39, wherein the liquid valve, the first gas valve, the second gas valve, or the light source are controlled based on information from the sensor, wherein the sensor is a liquid flow meter configured to measure a flow of a liquid between the first liquid tank and the liquid inlet.

Clause 66. The method of propagation of a plantlet of clause 39, wherein the liquid valve, the first gas valve, the second gas valve, or the light source are controlled based on information from the sensor, wherein the sensor is a microbial activity sensor configured to measure microbial activity in the replication capsule.

Clause 67. The method of propagation of a plantlet of clause 39, further comprising draining the first liquid mixture from the replication capsule to a waste tank coupled to the liquid outlet, wherein the waste tank is not coupled to the liquid inlet using a recirculation system.

Clause 68. The method of propagation of a plantlet of clause 39, further comprising: draining the first liquid mixture from the replication capsule to a sanitation tank coupled to the liquid outlet; sanitizing the first liquid mixture in the sanitation tank using a UV light, chemical treatment, or heat to form a sanitized liquid mixture in the sanitation tank; and recirculating the sanitized liquid mixture using a recirculation line coupling the sanitation tank to the liquid inlet.

The systems and methods of the preferred embodiment and variations thereof can be embodied and/or implemented at least in part as a machine configured to receive a computer-readable medium storing computer-readable instructions. The instructions are preferably executed by computer-executable components preferably integrated with the system and one or more portions of the processor in the plant tissue culture system and/or remote computing device. The computer-readable medium can be stored on any suitable computer-readable media such as RAMs, ROMs, flash memory, EEPROMs, optical devices (e.g., CD or DVD), hard drives, floppy drives, or any suitable device. The computer-executable component is preferably a general or application-specific processor, but any suitable dedicated hardware or hardware/firmware combination can alternatively or additionally execute the instructions.

As used in the description and claims, the singular form “a”, “an” and “the” include both singular and plural references unless the context clearly dictates otherwise. For example, the term “sensor” may include, and is contemplated to include, a plurality of sensors. At times, the claims and disclosure may include terms such as “a plurality,” “one or more,” or “at least one;” however, the absence of such terms is not intended to mean, and should not be interpreted to mean, that a plurality is not conceived.

The term “about” or “approximately,” when used before a numerical designation or range (e.g., to define a length or pressure), indicates approximations which may vary by (+) or (−) 5%, 1% or 0.1%. All numerical ranges provided herein are inclusive of the stated start and end numbers. The term “substantially” indicates mostly (i.e., greater than 50%) or essentially all of a device, substance, or composition.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a system or method consisting essentially of the elements as defined herein would not exclude other materials, features, or steps that do not materially affect the basic and novel characteristic(s) of the claimed disclosure. “Consisting of” shall mean that the devices, systems, and methods include the recited elements and exclude anything more than a trivial or inconsequential element or step. Embodiments defined by each of these transitional terms are within the scope of this disclosure.

As used herein, the terms “plantlet” and “young plant” are used to describe the produced product of culturing in vitro as well as within the devices and systems described herein, and are sometimes truncated to the term “plant.”

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A plant tissue culture apparatus comprising:
a replication capsule comprising a capsule body, liquid inlets, gas inlets, a gas outlet, and a liquid outlet, wherein an internal volume defined by the capsule body is configured to contain a plurality of plantlets;
a light source configured to illuminate the plurality of plantlets in the replication capsule;
a first gas tank coupled to the gas inlet and configured to contain a first gas;
a first liquid tank coupled to the liquid inlet, the first liquid tank configured to contain a first liquid mixture;
a nanobubble generator positioned between the first liquid tank and the liquid inlet;
a second gas tank configured to contain a second gas, wherein the second gas tank is coupled to the nanobubble generator, wherein the nanobubble generator is configured to generate bubbles comprising the second gas in the first liquid mixture;
a liquid valve between the first liquid tank and the liquid inlet;
a first gas valve between the first gas tank and the gas inlet;
a second gas valve between the second gas tank and the nanobubble generator; and
a processor coupled to the liquid valve, the first gas valve, the second gas valve, and the light source, wherein the processor is configured to control the liquid valve, the first gas valve, the second gas valve, and the light source based on predetermined parameters or in response to information from a sensor.

2. The plant tissue culture apparatus of claim 1, wherein the first liquid mixture comprises nutrients, and wherein the bubbles generated by the nanobubble generator comprise air with excess oxygen.

3. The plant tissue culture apparatus of claim 1, wherein the first gas comprises carbon dioxide, and wherein the second gas comprises oxygen.

4. The plant tissue culture apparatus of claim 1, further comprising a fan or air compressor coupled to a gas mixer, wherein the first gas tank is coupled to the gas mixer, and wherein the gas mixer is coupled to the gas inlet.

5. The plant tissue culture apparatus of claim 1, further comprising a liquid pump positioned between the first liquid tank and the liquid inlet.

6. The plant tissue culture apparatus of claim 1, wherein the first liquid tank and the liquid inlet are configured such that the first liquid mixture can be introduced into the replication capsule through the liquid inlet using a force of gravity.

7. The plant tissue culture apparatus of claim 1, further comprising a water source configured to provide filtered water, and a mixing tank, wherein:
the water source is coupled to the mixing tank,
the first liquid tank is coupled to the mixing tank,
the mixing tank is configured to dilute the first liquid mixture with the filtered water from the water source, and
the nanobubble generator is positioned between the mixing tank and the liquid inlet.

8. The plant tissue culture apparatus of claim 1, further comprising a second liquid tank coupled to the liquid inlet, wherein:
the second liquid tank is configured to contain a second liquid mixture,
the nanobubble generator is further positioned between the second liquid tank and the liquid inlet, and
the nanobubble generator is additionally configured to generate bubbles of the second gas in the second liquid mixture.

9. The plant tissue culture apparatus of claim 1, wherein the first liquid mixture comprises nutrients, wherein the second liquid mixture comprises microbiota, wherein the first gas comprises air with excess carbon dioxide, and wherein the second gas comprises air with excess oxygen.

10. The plant tissue culture apparatus of claim 1, further comprising a third liquid tank coupled to the liquid inlet, wherein the third liquid tank is configured to contain a third liquid mixture, wherein:
the nanobubble generator is further positioned between the third liquid tank and the liquid inlet,
the nanobubble generator is configured to generate bubbles of the second gas in the third liquid mixture, and
the third liquid mixture comprises a cleansing agent.

11. The plant tissue culture apparatus of claim 1, further comprising:
a mixing tank coupled to the liquid inlet;
a water source that is configured to provide filtered water coupled to the mixing tank; and
a plurality of liquid tanks coupled to the mixing tank, wherein the first liquid tank is one of a plurality of liquid tanks, wherein the plurality of liquid tanks each comprise a nutrient or a solution comprising microbiota;
wherein the nanobubble generator is further positioned between the mixing tank and the liquid inlet, and wherein the nanobubble generator is configured to generate bubbles of the second gas in liquid from the mixing tank.

12. The plant tissue culture apparatus of claim 1, wherein the processor is further configured to control one or more of an intensity of the light source, a spectrum of the light source, or a photoperiod of the light source.

13. The plant tissue culture apparatus of claim 1, wherein the replication capsule further comprises a membrane positioned between the gas inlet and the plurality of plantlets, and between the liquid inlet and the plurality of plantlets.

14. The plant tissue culture apparatus of claim 1, further comprising the sensor, wherein the processor is configured to control the liquid valve, the first gas valve, the second gas valve, and the light source based on information from the sensor, and wherein the sensor is one of: a temperature sensor configured to detect a temperature in the replication capsule, a humidity sensor configured to measure a humidity of the replication capsule, a vapor pressure deficit sensor configured to measure a vapor pressure deficit of the replication capsule, an electrical conductivity sensor configured to measure an electrical conductivity of the first liquid mixture, a pH sensor configured to measure a pH of the first liquid mixture, an air exchange rate sensor configured to measure an air exchange rate of the replication capsule, a carbon dioxide sensor configured to measure a carbon dioxide level of the replication capsule, an oxygen sensor configured to measure an oxygen level of the replication capsule, an ethylene sensor configured to measure an ethylene level of the replication capsule, a light sensor configured to measure light intensity level of illumination in the replication capsule, a liquid level sensor configured to measure a liquid level of the first liquid mixture in the first liquid tank or in the replication capsule, a gas flow meter configured to measure a flow of a gas between the first gas tank and the gas inlet, a liquid flow meter configured to measure a flow of a liquid between the first liquid tank and the liquid inlet, and a microbial activity sensor configured to measure microbial activity in the replication capsule.

15. The plant tissue culture apparatus of claim 1, wherein the sensor is a microbial activity sensor configured to measure microbial activity in the replication capsule, and wherein the processor is configured to control the liquid valve, the first gas valve, the second gas valve, or the light source based on information from the microbial activity sensor.

16. The plant tissue culture apparatus of claim 1, further comprising a waste tank coupled to the liquid outlet, wherein the waste tank is not coupled to the liquid inlet using a recirculation system.

17. The plant tissue culture apparatus of claim 1, further comprising a sanitation tank coupled to the liquid outlet and a recirculation line coupling the sanitation tank to the liquid inlet, wherein the sanitation tank comprises a UV light, chemical treatment, or heat to sterilize the liquid in the sanitation tank.

18. The plant tissue culture apparatus of claim 1, further comprising a plurality of replication capsules, wherein the plurality of replication capsules is coupled to the first gas tank and to the first liquid tank in a parallel configuration, in a series configuration, or in a combination parallel and series configuration.

19. The plant tissue culture apparatus of claim 1, wherein the replication capsule further comprises an antimicrobial coating.

20. The plant tissue culture apparatus of claim 1, further comprising a gas mixer, wherein the first gas tank and the second gas tank are coupled to the gas mixer, and wherein the gas mixer is coupled to the gas inlet and to the nanobubble generator.
